SEQUENCE LISTING (17 sequences)

SEQ ID NO: 1 - DNA, 1100 bp, Artificial Sequence
  Description of Artificial Sequence Synthetic polynucleotide
gctgcgcgtcagcgggatgtcgatgaaagatatcggcattatggatggtgacttgctggcagtgcataaaactcaggatgtacgtaacggtcaggtcgttgtcgcacgtattgatgacgaggttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattaggatccgtcgaccatggcggccgctcgagtcgacctgcagccaagctaattccgggcgaatttcttatgatttatgatttttattattaaataagttataaaaaaaataagtgtatacaaattttaaagtgactcttaggttttaaaacgaaaattcttattcttgagtaact

SEQ ID NO: 2 - DNA, 1073 bp, Artificial Sequence
  Description of Artificial Sequence Synthetic polynucleotide
gctgcgcgtcagcgggatgtcgatgaaagatatcggcattatggatggtgacttgctggcagtgcataaaactcaggatgtacgtaacggtcaggtcgttgtcgcacgtattgatgacgaggttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattcatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaggatccgtcgaccatggcggccgctcgagtcgacctgcagccaagctaattccgggcgaatttcttatgatttatgatttttattattaaataagttataaaaaaaataagtgtatacaaattttaaagtgactcttaggttttaaaacgaaaattcttattcttgagtaact

SEQ ID NO: 3 - DNA, 1109 bp, Artificial Sequence
  Description of Artificial Sequence Synthetic polynucleotide
gctgcgcgtcagcgggatgtcgatgaaagatatcggcattatggatggtgacttgctggcagtgcataaaactcaggatgtacgtaacggtcaggtcgttgtcgcacgtattgatgacgaggttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattcatgaacaagctccaacgcgaggccgtgatccgaaccgcgctcgaactgcttaacgacgtgggcatggaaggtctaacgacgcgccgactggctgagcgcctcggggtgcaacagccagcgctctactggcatttcaagaacaagcgtgcgttgctcgacgcacttgccgaagccatgctgacgataaatcacacgcattcgacgccaagggatgacgacgactggcgttcgttcctgaagggcaatgcatgcagttttcgacgggcgttgctcgcttatcgcgatggcgcgcgtattcatgccgggacgcggccagccgcgccgcagatggaaaaagccgacgcgcagcttcgcttcctttgcgatgctggcttttcggcaggtgacgcgacctatgcgttgatggcaatcagctacttcaccgtcggcgctgttcttgagcagcaagctagcgaggcagacgccgaggagcggggcgaagatcagttgaccacctcagcgtctacgatgccggcgcgcctacagagcgcgatgaaaatcgtctacgaaggcggtccggacgcggcattcgagcgaggcctggctctcatcatcggcggtcttgaaaaaatgaggctcactacgaacgacattgaggtgctgaagaatgttgacgaataggatccgtcgaccatggcggccgctcgagtcgacctgcagccaagctaattccgggcgaatttcttatgatttatgatttttattattaaataagttataaaaaaaataagtgtatacaaattttaaagtgactcttaggttttaaaacgaaaattcttattcttgagtaact

SEQ ID NO: 4 - DNA, 1106 bp, Artificial Sequence
  Description of Artificial Sequence Synthetic polynucleotide
gctgcgcgtcagcgggatgtcgatgaaagatatcggcattatggatggtgacttgctggcagtgcataaaactcaggatgtacgtaacggtcaggtcgttgtcgcacgtattgatgacgaggttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattcatggcacggctgaacagagaatcggttattgatgcggcactggaactgctgaatgagacagggattgacgggctgacgacccgcaagctggcgcagaagctgggaatagaacagccgacactttactggcatgtgaaaaataaacgggcgttactggatgcgctggcggtggagatcctggcgcgtcatcatgattattcactgcctgcggcgggggaatcctggcagtcatttctgcgcaataatgcaatgagtttccgccgggcgctgctgcgttaccgtgacggggcaaaagtgcacctcggcacccgccctgatgaaaaacagtatgatacggtggaaacccagttacgctttatgacagaaaacggcttttcactgcgcgacgggttatatgcgatttcagcggtcagtcattttacccttggtgccgtactggagcagcaggagcatactgccgccctgaccgaccgccctgcagcaccggacgaaaacctgccgccgctattgcgggaagcgctgcagattatggacagtgatgatggtgagcaggcctttctgcatggcctggagagcctgatccgggggtttgaggtgcagcttacggcactgttgcaaatagtcggtggtgataaacttatcatccccttttgctaggatccgtcgaccatggcggccgctcgagtcgacctgcagccaagctaattccgggcgaatttcttatgatttatgatttttattattaaataagttataaaaaaaataagtgtatacaaattttaaagtgactcttaggttttaaaacgaaaattcttattcttgagtaact

SEQ ID NO: 5 - DNA, 1085 bp, Artificial Sequence
  Description of Artificial Sequence Synthetic polynucleotide
gctgcgcgtcagcgggatgtcgatgaaagatatcggcattatggatggtgacttgctggcagtgcataaaactcaggatgtacgtaacggtcaggtcgttgtcgcacgtattgatgacgaggttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattcatggcacgactaagcttggacgacgtaatttcaatggcgctcaccctgctggacagcgaagggctagagggcttgactacgcgtaagctggcgcagtccctaaaaattgagcaaccgactctgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaataggatccgtcgaccatggcggccgctcgagtcgacctgcagccaagctaattccgggcgaatttcttatgatttatgatttttattattaaataagttataaaaaaaataagtgtatacaaattttaaagtgactcttaggttttaaaacgaaaattcttattcttgagtaact

SEQ ID NO: 6 - DNA, 1076 bp, Artificial Sequence
  Description of Artificial Sequence Synthetic polynucleotide
gctgcgcgtcagcgggatgtcgatgaaagatatcggcattatggatggtgacttgctggcagtgcataaaactcaggatgtacgtaacggtcaggtcgttgtcgcacgtattgatgacgaggttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattcatgaccaaactggacaagggcaccgtgatcgcggcggcgctagagctgttgaacgaggttggcatggacagcctgacgacgcggaagctcgctgaacgcctcaaggttcagcagcctgcgctttactggcatttccagaacaagcgagcgctgcttgatgcgctcgccgaggcgatgctggcggaacgccatacccgctcgctacccgaagagaatgaggactggcgggtgttcctgaaagagaatgccctgagcttcagaacggcgttgctctcttatcgggacggcgcgcgtatccatgccggcactcgaccgacagaaccgaattttggcaccgccgagacgcaaatacgctttctctgcgcggagggcttttgtccgaagcgcgccgtttgggcgctccgggcggtcagtcactatgtggtcggttccgttctcgagcagcaggcatctgatgccgatgagagagttccggacaggccagatgtgtccgagcaagcaccgtcgtccttcctgcacgatctgtttcacgagttggaaacagacggcatggatgctgcgttcaacttcggactcgacagcctcatcgctggtttcgagcggctgcgttcatctacaacagattaggatccgtcgaccatggcggccgctcgagtcgacctgcagccaagctaattccgggcgaatttcttatgatttatgatttttattattaaataagttataaaaaaaataagtgtatacaaattttaaagtgactcttaggttttaaaacgaaaattcttattcttgagtaact

SEQ ID NO: 7 - DNA, 1073 bp, Artificial Sequence
  Description of Artificial Sequence Synthetic polynucleotide
gctgcgcgtcagcgggatgtcgatgaaagatatcggcattatggatggtgacttgctggcagtgcataaaactcaggatgtacgtaacggtcaggtcgttgtcgcacgtattgatgacgaggttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattcatggcaaagctagataaagaacaagttattgatgatgcgttgattttacttaatgaagttggtattgaaggattaacaacgcgtaacgtggcgcaaaaaataggtgtggaacaacccacattgtattggcatgtaaaaaataaacgcgctttgttagatgcattagcagaaactattttgcaaaagcaccatcatcatgttttgccattgccgaatgaaacatggcaggactttttgcgaaataacgcgaaaagcttccgccaagccttattaatgtatcgtgatggtggcaaaattcatgcgggaacacgcccctctgaaagtcaatttgagacatcagaacagcaactacagtttttgtgtgatgctgggtttagtctatctcaagccgtgtatgcattaagctctattgcgcattttacattaggctccgtactggaaactcaagagcatcaagaaagccaaaaagagcgtgaaaaagtagagacggatactgttgcctatccgccattattaacccaagccgttgcaattatggatagtgataatggtgatgctgcatttttgtttgtccttgatgtgatgatctctggacttgaaacagtattaaagagcgctaaataggatccgtcgaccatggcggccgctcgagtcgacctgcagccaagctaattccgggcgaatttcttatgatttatgatttttattattaaataagttataaaaaaaataagtgtatacaaattttaaagtgactcttaggttttaaaacgaaaattcttattcttgagtaact

SEQ ID NO: 8 - DNA, 651 bp, Artificial Sequence
  Description of Artificial Sequence Synthetic polynucleotide
atgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattag

SEQ ID NO: 9 - DNA, 624 bp, Artificial Sequence
  Description of Artificial Sequence Synthetic polynucleotide
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttag

SEQ ID NO: 10 - DNA, 660 bp, Artificial Sequence
  Description of Artificial Sequence Synthetic polynucleotide
atgaacaagctccaacgcgaggccgtgatccgaaccgcgctcgaactgcttaacgacgtgggcatggaaggtctaacgacgcgccgactggctgagcgcctcggggtgcaacagccagcgctctactggcatttcaagaacaagcgtgcgttgctcgacgcacttgccgaagccatgctgacgataaatcacacgcattcgacgccaagggatgacgacgactggcgttcgttcctgaagggcaatgcatgcagttttcgacgggcgttgctcgcttatcgcgatggcgcgcgtattcatgccgggacgcggccagccgcgccgcagatggaaaaagccgacgcgcagcttcgcttcctttgcgatgctggcttttcggcaggtgacgcgacctatgcgttgatggcaatcagctacttcaccgtcggcgctgttcttgagcagcaagctagcgaggcagacgccgaggagcggggcgaagatcagttgaccacctcagcgtctacgatgccggcgcgcctacagagcgcgatgaaaatcgtctacgaaggcggtccggacgcggcattcgagcgaggcctggctctcatcatcggcggtcttgaaaaaatgaggctcactacgaacgacattgaggtgctgaagaatgttgacgaatag

SEQ ID NO: 11 - DNA, 657 bp, Artificial Sequence
  Description of Artificial Sequence Synthetic polynucleotide
atggcacggctgaacagagaatcggttattgatgcggcactggaactgctgaatgagacagggattgacgggctgacgacccgcaagctggcgcagaagctgggaatagaacagccgacactttactggcatgtgaaaaataaacgggcgttactggatgcgctggcggtggagatcctggcgcgtcatcatgattattcactgcctgcggcgggggaatcctggcagtcatttctgcgcaataatgcaatgagtttccgccgggcgctgctgcgttaccgtgacggggcaaaagtgcacctcggcacccgccctgatgaaaaacagtatgatacggtggaaacccagttacgctttatgacagaaaacggcttttcactgcgcgacgggttatatgcgatttcagcggtcagtcattttacccttggtgccgtactggagcagcaggagcatactgccgccctgaccgaccgccctgcagcaccggacgaaaacctgccgccgctattgcgggaagcgctgcagattatggacagtgatgatggtgagcaggcctttctgcatggcctggagagcctgatccgggggtttgaggtgcagcttacggcactgttgcaaatagtcggtggtgataaacttatcatccccttttgctag

SEQ ID NO: 12 - DNA, 636 bp, Artificial Sequence
  Description of Artificial Sequence Synthetic polynucleotide
atggcacgactaagcttggacgacgtaatttcaatggcgctcaccctgctggacagcgaagggctagagggcttgactacgcgtaagctggcgcagtccctaaaaattgagcaaccgactctgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaatag

SEQ ID NO: 13 - DNA, 627 bp, Artificial Sequence
  Description of Artificial Sequence Synthetic polynucleotide
atgaccaaactggacaagggcaccgtgatcgcggcggcgctagagctgttgaacgaggttggcatggacagcctgacgacgcggaagctcgctgaacgcctcaaggttcagcagcctgcgctttactggcatttccagaacaagcgagcgctgcttgatgcgctcgccgaggcgatgctggcggaacgccatacccgctcgctacccgaagagaatgaggactggcgggtgttcctgaaagagaatgccctgagcttcagaacggcgttgctctcttatcgggacggcgcgcgtatccatgccggcactcgaccgacagaaccgaattttggcaccgccgagacgcaaatacgctttctctgcgcggagggcttttgtccgaagcgcgccgtttgggcgctccgggcggtcagtcactatgtggtcggttccgttctcgagcagcaggcatctgatgccgatgagagagttccggacaggccagatgtgtccgagcaagcaccgtcgtccttcctgcacgatctgtttcacgagttggaaacagacggcatggatgctgcgttcaacttcggactcgacagcctcatcgctggtttcgagcggctgcgttcatctacaacagattag

SEQ ID NO: 14 - DNA, 624 bp, Artificial Sequence
  Description of Artificial Sequence Synthetic polynucleotide
atggcaaagctagataaagaacaagttattgatgatgcgttgattttacttaatgaagttggtattgaaggattaacaacgcgtaacgtggcgcaaaaaataggtgtggaacaacccacattgtattggcatgtaaaaaataaacgcgctttgttagatgcattagcagaaactattttgcaaaagcaccatcatcatgttttgccattgccgaatgaaacatggcaggactttttgcgaaataacgcgaaaagcttccgccaagccttattaatgtatcgtgatggtggcaaaattcatgcgggaacacgcccctctgaaagtcaatttgagacatcagaacagcaactacagtttttgtgtgatgctgggtttagtctatctcaagccgtgtatgcattaagctctattgcgcattttacattaggctccgtactggaaactcaagagcatcaagaaagccaaaaagagcgtgaaaaagtagagacggatactgttgcctatccgccattattaacccaagccgttgcaattatggatagtgataatggtgatgctgcatttttgtttgtccttgatgtgatgatctctggacttgaaacagtattaaagagcgctaaatag

SEQ ID NO: 15 - DNA, 1517 bp, Artificial Sequence
  Description of Artificial Sequence Synthetic polynucleotide
atacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagttatttatataattcatccataccaccagttgaatgtctaccttcagctctttcatattgttcaacaatagtataatcttcattatgtgaagtaatatccaatttaatattaacattataagcacctggtaattgaactggttttttagctttataagtagttttaacttcagcatcataatgaccaccatcttttaatttcaatctttgtttaatttcaccttttaaagcaccatcttctggatacattctttctgatgaagcttcccaacccatagtttttttttgcataactggaccatctgatggaaaattagtacctctcaatttaactttataaataaattcaccatcttgtaatgatgaatcttgagtaacagtaacaacaccaccatcttcaaaattcataactctttcccatttaaaaccttctggaaatgacaattttaaataatctggaatatcagctggatgtttaacataagcttttgaaccatacataaattgtggtgacaaaatatcccaagcaaatggtaatggaccacctttagtaactttcaatttagcagtttgagtaccttcatatggtctaccttcaccttcaccttcaatttcaaattcatgaccattaactgaaccttccatatgaactttaaatctcataaattctttaataatagccatattatcttcttcaccttttgaaaccattatagttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcccctcgacgtactgtacatataaccactggttttatatacagcagtactgtacatataaccactggttttatatacagcagtcgacgtactgtacatataaccactggttttatatacagcagtactggacatataaccactggttttatatacagcagtcgaggtaagattagatatgagctccagcttttgttccctttagtgagggttaattgcgcgcttggcgtaatcatggtcatagctgtttcctgtgtgaaattgttatccgctcacaattccacacaacata

SEQ ID NO: 16 - DNA, 1117 bp, Artificial Sequence
  Description of Artificial Sequence Synthetic polynucleotide
ttatttatataattcatccataccaccagttgaatgtctaccttcagctctttcatattgttcaacaatagtataatcttcattatgtgaagtaatatccaatttaatattaacattataagcacctggtaattgaactggttttttagctttataagtagttttaacttcagcatcataatgaccaccatcttttaatttcaatctttgtttaatttcaccttttaaagcaccatcttctggatacattctttctgatgaagcttcccaacccatagtttttttttgcataactggaccatctgatggaaaattagtacctctcaatttaactttataaataaattcaccatcttgtaatgatgaatcttgagtaacagtaacaacaccaccatcttcaaaattcataactctttcccatttaaaaccttctggaaatgacaattttaaataatctggaatatcagctggatgtttaacataagcttttgaaccatacataaattgtggtgacaaaatatcccaagcaaatggtaatggaccacctttagtaactttcaatttagcagtttgagtaccttcatatggtctaccttcaccttcaccttcaatttcaaattcatgaccattaactgaaccttccatatgaactttaaatctcataaattctttaataatagccatattatcttcttcaccttttgaaaccattatagttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcccctcgacgtactgtacatataaccactggttttatatacagcagtactgtacatataaccactggttttatatacagcagtcgacgtactgtacatataaccactggttttatatacagcagtactggacatataaccactggttttatatacagcagtcgaggtaagattagatatg

SEQ ID NO: 17 - DNA, 798 bp, Artificial Sequence
  Description of Artificial Sequence Synthetic polynucleotide
atgggtgctcctccaaaaaagaagagaaaggtagctggtatcaataaagatatcgaggagtgcaatgccatcattgagcagtttatcgactacctgcgcaccggacaggagatgccgatggaaatggcggatcaggcgattaacgtggtgccgggcatgacgccgaaaaccattcttcacgccgggccgccgatccagcctgactggctgaaatcgaatggttttcatgaaattgaagcggatgttaacgataccagcctcttgctgagtggagatgcctcctacccttatgatgtgccagattatgcctctcccgaattgatcagtctgattgcggcgttagcggtagatcgcgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgggtacggacgatcgcgtaacgtgggtgaagtcggtggatgaagccatcgcggcgtgtggtgacgtaccagaaatcatggtgattggcggcggtcgcgtttatgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcggtaa

(19) United States
(12) Patent Application Publication
(10) Pub. No.: US 2022/0112470 A1
(43) Pub. Date: Apr. 14, 2022

(54) SYSTEMS AND METHODS FOR METABOLIC ENGINEERING

(71) Applicant: THE TRUSTEES OF COLUMBIA UNIVERSITY IN THE CITY OF NEW YORK, New York, NY (US)

(72) Inventors: Virginia W. CORNISH, New York, NY (US); Ehud HERBST, New York, NY (US)

(73) Assignee: THE TRUSTEES OF COLUMBIA UNIVERSITY IN THE CITY OF NEW YORK, New York, NY (US)

(21) Appl. No.: 17/556,708

(22) Filed: Dec. 20, 2021

Related U.S. Application Data
(63) Continuation of application No. PCT/US2020/039005, filed on Jun. 22, 2020.
(60) Provisional application No. 62/864,915, filed on Jun. 21, 2019.

(51) Int. Cl. C12N 9/06; C12Q 1/6897; C12N 15/81

(52) U.S. Cl. CPC C12N 9/003; C12Y 105/01003; C07K 2319/71; C12N 15/81; C07K 2319/80; C12Q 1/6897

(57) ABSTRACT

The presently disclosed subject matter provides high-throughput assays, e.g., yeast three-hybrid assays, for detecting the presence of a molecule, e.g., therapeutic molecule. In certain embodiments, the presently disclosed subject matter provides genetically-engineered cells and assays for detecting tetracycline or derivatives thereof and kits for performing such assays.

### GRANT INFORMATION

This invention was made with government support under grant number R01-AI110794 awarded by the National Institutes of Health (NIH). The government has certain rights in the invention.

### SEQUENCE LISTING

The instant application contains a Sequence Listing which has been submitted electronically in ASCII format and is hereby incorporated by reference in its entirety. Said ASCII copy, created on Dec. 20, 2021, is named 070050_6586 SL.txt and is 23,734 bytes in size. The Sequence Listing does not extend beyond the scope of the specification and thus does not contain new matter.

### CROSS-REFERENCE TO RELATED APPLICATIONS

This application is a continuation of International Patent Application No. PCT/US 2020/039005 filed Jun. 22, 2020, which claims priority to U.S. Provisional Application No. 62/864,915, filed on Jun. 21, 2019, the contents of which are incorporated by reference in their entirety, and to which priority is claimed.

### BACKGROUND

Metabolic engineering can be an environment friendly approach for production of a wide range of organic chemicals. Certain metabolic engineering techniques can produce chemicals from readily available and renewable carbon sources, resulting in the reduced use of organic solvents and reagents. For efficient metabolic engineering, high-throughput and versatile strain construction techniques can be required to assay vast libraries of strains producing desirable chemicals. However, certain metabolic engineering techniques can be low-throughput or non-universal, thereby limiting implementation.

Therefore, there is a need for versatile, general and readily implemented high-throughput assays to assay the genetically-engineered cells used for metabolic engineering.

### SUMMARY

The disclosed subject matter provides genetically-engineered cells, assays and kits for detecting the production of a molecule, e.g., a therapeutic molecule. For example, but not by way of limitation, the present disclosure provides yeast-based assays, e.g., yeast three-hybrid assays, for detecting the production of a molecule, e.g., therapeutic molecule, e.g., tetracycline or a tetracycline analogue.

In a first aspect, the present disclosure provides a genetically-engineered cell that expresses a first fusion protein that includes (i) a DNA-binding domain and (ii) a protein or fragment thereof that binds to a molecule, e.g., therapeutic molecule. The genetically-engineered cell can further express a second fusion protein that includes (i) an activation domain and (ii) a protein or fragment thereof that binds to methotrexate.

In certain embodiments, the DNA-binding domain of the first fusion protein is LexA. In certain embodiments, the protein or fragment thereof of the first fusion protein that binds to a molecule, e.g., therapeutic molecule, is a tetracycline receptor (TetR), e.g., TetR(A), TetR(B), TetR(C), TetR(D), TetR(E), TetR(G) or TetR(H). In certain embodiments, the first fusion protein is encoded by a nucleotide sequence that is at least about 90% homologous to a sequence of any one of SEQ ID NOs: 1-7. In certain embodiments, the protein or fragment thereof that binds to a therapeutic molecule is encoded by a nucleotide sequence that is at least about 90% homologous to a sequence of any one of SEQ ID NOs: 8-14. In certain embodiments, the activation domain of the second fusion protein is B42. In certain embodiments, the protein or fragment thereof that binds to methotrexate is dihydrofolate reductase (DHFR). In certain embodiments, the second fusion protein is encoded by a nucleotide sequence that is at least about 90% homologous to the sequence of SEQ ID NO: 17.

In certain embodiments, the genetically-engineered cell can further include a nucleic acid that includes (i) a DNA-binding site that binds to the DNA-binding domain of the first fusion protein and (ii) a reporter gene. In certain embodiments, the reporter gene is a conditionally-essential gene, e.g., URA3, or encodes LacZ or a fluorescent protein. In certain embodiments, the nucleic acid comprises a nucleotide sequence that is at least about 90% homologous to a sequence of any one of SEQ ID NOs: 15-16.

In certain embodiments, the genetically-engineered cell can further include a chemical inducer of dimerization (CID). In certain embodiments, the CID includes (i) a ligand that binds to the protein or fragment thereof of the first fusion protein that binds to the molecule, e.g., therapeutic molecule, and (ii) methotrexate.

In certain embodiments, the dimerization of the first fusion protein and the second fusion protein induces expression of the reporter gene.

In a further aspect, the present disclosure provides an assay system, e.g., for detecting the presence of a molecule, e.g., therapeutic molecule. In certain embodiments, the assay system includes a genetically-engineered cell disclosed herein. For example, but not by way of limitation, the genetically-engineered cell expresses a first fusion protein and a second fusion protein as disclosed herein. In certain embodiments, the assay system further includes a CID as disclosed herein.

In another aspect, the present disclosed subject matter provides assays for detecting the presence of a molecule, e.g., therapeutic molecule. In certain embodiments, the assay includes contacting a genetically-engineered cell disclosed herein with a CID. In certain embodiments, the CID includes (i) a ligand that binds to the protein or fragment thereof of the first fusion protein that binds to the molecule, e.g., therapeutic molecule, and (ii) methotrexate. In certain embodiments, the assay can further include contacting the genetically-engineered cell with a sample. In certain embodiments, the assay can further include detecting the expression of the reporter gene.

In certain embodiments, the CID dimerizes the first and second fusion proteins to induce expression of the reporter gene. In certain embodiments, the molecule, e.g., therapeutic molecule, competes with the CID for binding to the protein or fragment thereof of the first fusion protein that binds to the molecule, e.g., therapeutic molecule, e.g., blocks dimerization of the first fusion protein and the second fusion protein. In certain embodiments, reduced expression of the reporter gene as compared to a reference control indicates the presence of the molecule, e.g., therapeutic molecule, in the sample. In certain embodiments, the sample is supernatant from a culture of a cell expressing, e.g., engineered to express, the molecule, e.g., therapeutic molecule. In certain embodiments, the ligand of the CID that binds to the protein or fragment thereof of the first fusion protein that binds to the molecule, e.g., therapeutic molecule, is a tetracycline derivative.

In certain embodiments, the DNA-binding domain of the first fusion protein is LexA or a DNA-binding domain thereof and/or the protein or fragment thereof of the first fusion protein that binds to a molecule, e.g., therapeutic molecule, is a tetracycline receptor (TetR) or a variant thereof. In certain embodiments, the activation domain of the second fusion protein is B42 or an activation domain thereof and/or the protein or fragment thereof of the second fusion protein that binds to methotrexate is dihydrofolate reductase (DHFR). In certain embodiments, the DNA-binding site of the nucleic acid is a binding site for LexA or a DNA-binding domain thereof and/or the reporter gene of the nucleic acid is a conditionally-essential gene or encodes LacZ or a fluorescent protein.

The presently disclosed subject matter further provides a kit for detecting the presence of a molecule, e.g., therapeutic molecule. In certain embodiments, the kit can include a genetically-engineered cell that includes a nucleic acid that has a (i) a DNA-binding site and (ii) a reporter gene. Alternatively or additionally, a kit of the present disclosure can include a cell and a nucleic acid that has a (i) a DNA-binding site and (ii) a reporter gene. In certain embodiments, a kit of the present disclosure can include a nucleic acid encoding a first fusion protein that includes (i) a DNA-binding domain that binds to the DNA-binding site and (ii) a protein or fragment thereof that binds to a molecule, e.g., therapeutic molecule, and/or a nucleic acid encoding a second fusion protein that includes (i) an activation domain and (ii) a protein or fragment thereof that binds to methotrexate. In certain embodiments, a kit of the present disclosure can include a chemical inducer of dimerization (CID), wherein the CID includes (i) a ligand that binds to the protein or fragment thereof of the first fusion protein that binds to the molecule, e.g., therapeutic molecule, and (ii) methotrexate.

In certain embodiments, the genetically-engineered cell for use in the disclosed assays is selected from the group consisting of a mammalian cell, a plant cell, a bacterial cell and a fungal cell. In certain embodiments, the genetically-engineered cell is a yeast cell, e.g., Saccharomyces cerevisiae.

In certain embodiments, the molecule, e.g., therapeutic molecule, is selected from the group consisting of a peptide, a protein or portion thereof and a small molecule. In certain embodiments, the molecule, e.g., therapeutic molecule, is a small molecule. For example, but not by way of limitation, the small molecule is tetracycline or a derivative thereof.

### BRIEF DESCRIPTION OF THE FIGURES

FIG. 1. Detection of a target molecule biosynthesis by the yeast three hybrid (Y3H) system. Low/no production of a target molecule is detected by high reporter gene expression (left). High production titers of the target molecule outcompete the chemical inducer of dimerization (CID) from the fusion protein receptor and are detected by lower gene expression (right). DBD=DNA-binding domain (e.g., LexA); AD=activation domain (e.g., B42); PR=protein receptor (e.g., TetR); DHFR=dihydrofolate reductase.

FIG. 2A-2C. Characterization of the dynamic range of the Y3H assay for tetracyclines in (A) varying concentrations of the Min-Mtx CID, (B) varying TetR classes and (C) varying target molecule structure and concentrations. LacZ was used as a reporter gene. Miller units were calculated as (1000×OD420)/(well volume (mL)× reaction time (min)×OD600).⁵⁶ “+TetR” and “−TetR” are strains HL-260-5 and HL-260-7 encoding LexA-TetR(G) and LexA, respectively; Error bars represent the standard error of the mean (SEM) from three biological replicates.

FIG. 3A-3C. Modularity of the Y3H assay enables both screening and selecting for biosynthesis of target tetracyclines by enabling (A) a growth assay, (B) a colorimetric assay, and a (C) fluorometric assay. (A) Yeast growth, (B) LacZ transcription and (C) mCherry transcription are dependent on doxycycline (Dox) in the presence but not in the absence of the CID. CID, Dox concentrations=(A) 25 μM, 5 μM; (B) 25 μM, 0.2 μM; (C) 10 μM, 1 μM. Error bars represent the standard error of the mean (SEM) from three biological replicates. Miller units were calculated as (1000×OD420)/(well volume (mL)×reaction time (min)×OD600).⁵⁶ mCherry (620 nm)/OD600 was calculated by dividing mCherry fluorescence (620 nm, λex=588 nm) by OD600.

FIG. 4A-4C. Differentiation of a producer and a nonproducer strain of a target molecule by the Y3H assay for tetracyclines, demonstrating the applicability of the Y3H assay to metabolic engineering. (A) Workflow for screening colonies for production of a target molecule using the Y3H assay. (B) The Y3H assay for tetracyclines differentiates between the supernatants of TAN-1612 producer and TAN-1612 nonproducer cultured in shake flasks. (C) The Y3H assay for tetracyclines differentiates the supernatants of TAN-1612 producer colonies from a nonproducing population cultured in a 96-well plate. The nonproducer strain does not encode AdaA, the polyketide synthase of the TAN-1612 biosynthetic pathway, while the producer strain encodes the complete pathway (Tables 1 and 2). The concentration of TAN-1612 in the Y3H assay well from the supernatant and from the purified sample is ˜0.3 and 0.5 μM, respectively. mCherry (620 nm)/OD600 was calculated by dividing mCherry fluorescence (620 nm, λex=588 nm) by OD600. Error bars represent the standard error of the mean (SEM) from three biological replicates of the Y3H strain and two biological replicates of the TAN-1612 producer/nonproducer.

FIG. 5. LacZ readout of strain harboring plasmid encoding for LexA-TetR (PBA-8) and strain without the plasmid (PBA-5). Only the strain with the LexA-TetR fusion protein responds to the CID. Addition of Dox lowers reporter gene expression only in the strain with LexA-TetR in the presence of the CID. Concentrations of CID and Dox are 25 μM and 200 nM, respectively. Miller units were calculated as (1000*OD420)/(well-volume(mL)*reaction-time(min)*OD600) (see Zhang, X., and Bremer, H. (1995) Control of the Escherichia coli rrnB P1 promoter strength by ppGpp, J. Biol. Chem. 270, 11181-11189). Error bars represent the standard error of the mean (s.e.m) from three biological replicates.

FIG. 6. A standard curve for TAN-1612 quantification in cultures. Purified TAN-1612 was analyzed at different concentrations on a separation gradient of 5% to 95% MeCN in supercritical CO₂ over 5.5 minutes. Absorption between 390 nm to 410 nm was integrated at retention times of 0.87-1.50 min, which corresponds to TAN-1612 (MS (AI−): m/z calc. for C₂₁H₁₇O₉—: 413.09; found: 413.4 [M−H]−; MS (EI−): m/z calc. for C₂₁H₁₇O₉—: 413.09; found: 413.4 [M−H]−).

### DETAILED DESCRIPTION

The present disclosure relates to the use of a high throughput assay for detecting the presence of a molecule, e.g., therapeutic molecule. For example, but not by way of limitation, the present disclosure provides yeast three-hybrid systems that include genetically-engineered cells that communicate with each other, and kits thereof. In particular, a yeast three-hybrid system described herein is generally useful for determining whether a cell produces a molecule, e.g., a therapeutic, e.g., a small molecule therapeutic, or whether a cell produces a molecule, e.g., therapeutic, at a greater level than a second cell.

For clarity, but not by way of limitation, the detailed description of the presently disclosed subject matter is divided into the following subsections:

I. Definitions;

II. Cells;

III. Assays;

IV. Molecules; and

V. Kits.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and still up to 1% of a given value. Alternatively, particularly with respect to biological systems or processes, the term can mean within an order of magnitude, within 5-fold, and within 2-fold, of a value.

The term “expression” or “expresses,” as used herein, refer to transcription and translation occurring within a cell, e.g., yeast cell. The level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, “polypeptide” refers generally to peptides and proteins having about three or more amino acids. The polypeptides can be endogenous to the cell, or can be exogenous, meaning that they are heterologous, i.e., foreign, to the cell being utilized. In certain embodiments, synthetic peptides are used, e.g., those which are directly secreted into the medium.

The term “protein” is meant to refer to a sequence of amino acids for which the chain length is sufficient to produce the higher levels of tertiary and/or quaternary structure. This is to distinguish from “peptides” that typically do not have such structure. Typically, the protein herein will have a molecular weight of at least about 15-100 kD, e.g., closer to about 15 kD. In certain embodiments, a protein can include at least about 50, about 60, about 70, about 80, about 90, about 100, about 200, about 300, about 400 or about 500 amino acids. Examples of proteins encompassed within the definition herein include all proteins, and, in general proteins that contain one or more disulfide bonds, including multi-chain polypeptides comprising one or more inter- and/or intrachain disulfide bonds. In certain embodiments, proteins can include other post-translation modifications including, but not limited to, glycosylation and lipidation. See, e.g., Prabakaran et al., WIREs Syst Biol Med (2012), which is incorporated herein by reference in its entirety.

As used herein the term “amino acid,” “amino acid monomer” or “amino acid residue” refers to organic compounds composed of amine and carboxylic acid functional groups, along with a side-chain specific to each amino acid. In particular, alpha- or α-amino acid refers to organic compounds in which the amine (—NH2) is separated from the carboxylic acid (—COOH) by a methylene group (—CH2), and a side-chain specific to each amino acid connected to this methylene group (—CH2) which is alpha to the carboxylic acid (—COOH). Different amino acids have different side chains and have distinctive characteristics, such as charge, polarity, aromaticity, reduction potential, hydrophobicity, and pKa. Amino acids can be covalently linked to form a polymer through peptide bonds by reactions between the carboxylic acid group of the first amino acid and the amine group of the second amino acid. Amino acid in the sense of the disclosure refers to any of the twenty plus naturally occurring amino acids, non-natural amino acids, and includes both D and L optical isomers.

The term “nucleic acid,” “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of a fusion protein of the disclosure in vitro and/or in vivo, e.g., in a yeast cell. Such DNA (e.g., cDNA) or RNA (e.g., mRNA) vectors, can be unmodified or modified. For example, mRNA can be chemically modified to enhance the stability of the RNA vector and/or expression of the encoded molecule.

As used herein, the term “vector” refers to a nucleic acid molecule capable of transporting another nucleic acid to which it has been linked.

As used herein, the term “recombinant cell” refers to cells which have some genetic modification from the original parent cells from which they are derived. Such cells can also be referred to as “genetically-engineered cells.” Such genetic modification can be the result of an introduction of a heterologous gene (or nucleic acid) for expression of the gene product, e.g., a recombinant protein, e.g., a therapeutic.

As used herein, the term “recombinant protein” refers generally to peptides and proteins. Such recombinant proteins are “heterologous,” i.e., foreign to the cell being utilized, such as a heterologous secretory peptide produced by a yeast cell.

As used herein, “sequence identity” or “identity” in the context of two polynucleotide or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

As used herein, “percentage of sequence identity” means the value determined by comparing two optimally aligned sequences over a comparison window, wherein the portion of the polynucleotide sequence in the comparison window can include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As understood by those skilled in the art, determination of percent identity between any two sequences can be accomplished using certain well-known mathematical algorithms. Non-limiting examples of such mathematical algorithms are the algorithm of Myers and Miller, the local homology algorithm of Smith et al.; the homology alignment algorithm of Needleman and Wunsch; the search-for-similarity-method of Pearson and Lipman; the algorithm of Karlin and Altschul, modified as in Karlin and Altschul. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons.

As used herein, “reference sequence” is a defined sequence used as a basis for sequence comparison. A reference sequence can be a subset or the entirety of a specified sequence; for example, as a segment of a full-length protein or protein fragment. A reference sequence can be, for example, a sequence identifiable in a database such as GenBank and UniProt and others identifiable to those skilled in the art.

The term “operative connection” or “operatively linked,” as used herein, with regard to regulatory sequences of a gene indicate an arrangement of elements in a combination enabling production of an appropriate effect. With respect to genes and regulatory sequences, an operative connection indicates a configuration of the genes with respect to the regulatory sequence allowing the regulatory sequences to directly or indirectly increase or decrease transcription or translation of the genes. In particular, in certain embodiments, regulatory sequences directly increasing transcription of the operatively linked gene, comprise promoters typically located on a same strand and upstream on a DNA sequence (towards the 5′ region of the sense strand), adjacent to the transcription start site of the genes whose transcription they initiate. In certain embodiments, regulatory sequences directly increasing transcription of the operatively linked gene or gene cluster comprise enhancers that can be located more distally from the transcription start site compared to promoters, and either upstream or downstream from the regulated genes, as understood by those skilled in the art. Enhancers are typically short (50-1500 bp) regions of DNA that can be bound by transcriptional activators to increase transcription of a particular gene. Typically, enhancers can be located up to 1 Mbp away from the gene, upstream or downstream from the start site.

The term “secretable,” as used herein, means able to be secreted, wherein secretion in the present disclosure generally refers to transport or translocation from the interior of a cell, e.g., within the cytoplasm or cytosol of a cell, to its exterior, e.g., outside the plasma membrane of the cell. Secretion can include several procedures, including various cellular processing procedures such as enzymatic processing of the peptide.

As would be understood by those skilled in the art, the term “codon optimization,” as used herein, refers to the introduction of synonymous mutations into codons of a protein-coding gene in order to improve protein expression in expression systems of a particular organism, such as a cell of a species of the phylum Ascomycota, in accordance with the codon usage bias of that organism. The term “codon usage bias” refers to differences in the frequency of occurrence of synonymous codons in coding DNA. The genetic codes of different organisms are often biased towards using one of the several codons that encode a same amino acid over others—thus using the one codon with, a greater frequency than expected by chance. Optimized codons in microorganisms, such as Saccharomyces cerevisiae, reflect the composition of their respective genomic tRNA pool. The use of optimized codons can help to achieve faster translation rates and high accuracy.

In the field of bioinformatics and computational biology, many statistical methods have been discussed and used to analyze codon usage bias. Methods such as the ‘frequency of optimal codons’ (Fop), the Relative Codon Adaptation (RCA) or the ‘Codon Adaptation Index’ (CAI) are used to predict gene expression levels, while methods such as the ‘effective number of codons’ (Nc) and Shannon entropy from information theory are used to measure codon usage evenness. Multivariate statistical methods, such as correspondence analysis and principal component analysis, are widely used to analyze variations in codon usage among genes. There are many computer programs to implement the statistical analyses enumerated above, including CodonW, GCUA, INCA, and others identifiable by those skilled in the art. Several software packages are available online for codon optimization of gene sequences, including those offered by companies such as GenScript, EnCor Biotechnology, Integrated DNA Technologies, ThermoFisher Scientific, among others known those skilled in the art. Those packages can be used in providing fusion protein genetic molecular components with codon ensuring optimized expression in assay systems as will be understood by a skilled person.

The terms “binding” or “bind,” as used herein, refer to the connecting or uniting of two or more components by a interaction, bond, link, force or tie in order to keep two or more components together, which encompasses either direct or indirect binding where, for example, a first component is directly bound to a second component, or one or more intermediate molecules are disposed between the first component and the second component. Exemplary bonds comprise covalent bond, ionic bond, van der Waals interactions and other bonds identifiable by a skilled person. In certain embodiments, the binding can be direct, such as the production of a polypeptide scaffold that directly binds to a scaffold-binding element of a protein. In certain embodiments, the binding can be indirect, such as the co-localization of multiple protein elements on one scaffold. In certain embodiments, binding of a component with another component can result in sequestering the component, thus providing a type of inhibition of the component. In certain embodiments, binding of a component with another component can change the activity or function of the component, as in the case of allosteric or other interactions between proteins that result in conformational change of a component, thus providing a type of activation of the bound component. Examples described herein include, without limitation, binding of a DNA-binding domain of a fusion protein to a DNA-binding site.

The term “selectively activates,” as used herein, refers to the ability of a ligand to activate a receptor, e.g., preferentially interact with, in the presence of other different receptors.

The term “reportable component,” as used herein, indicates a component capable of detection in one or more systems and/or environments.

The terms “detect” or “detection,” as used herein, indicates the determination of the existence and/or presence of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

As used herein, the term “therapeutic molecule” includes any small molecule, protein and peptide that can be administered to a subject and provide a therapeutic effect, such as reduce, alleviate, or eliminate symptoms or pathologies of a disease or disorder. In certain embodiments, a therapeutic molecule stimulates or reduces an immune response to an antigen or allergen.

“Pharmaceutically acceptable carrier” as used herein refers to a pharmaceutically acceptable material, composition, or vehicle that is involved in carrying or transporting a compound or composition of interest from one tissue, organ, or portion of the body to another tissue, organ, or portion of the body. For example, but not by way of limitation, the carrier can be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it can come in contact, meaning that it must not carry a risk of toxicity, irritation, allergic response, immunogenicity, or any other complication that excessively outweighs its therapeutic benefits.

As used herein the term “subject” refers to any animal (e.g., a mammal), including, but not limited to, humans, non-human primates, rodents, and the like, which is to be recipient of a particular treatment.

The term “derived” or “derive” is used herein to mean to obtain from a specified source.

As used herein, the term “fusion protein” refers to a protein that includes all or a portion of a protein that is linked, e.g., at the N-terminus or C-terminus, to a second protein or a portion of the second protein.

As used herein, the term “chemical inducer of dimerization” refers to a heterodimeric small molecule that can dimerize two proteins or fragments of such proteins efficiently in vivo.

A “conditionally-essential gene,” as used herein, refers to a gene that is essential for growth and/or survival under certain conditions but not others, e.g., in the absence of an essential media component or in the presence of a toxic precursor. A non-limiting example of a conditionally-essential gene is URA3.

### II. Cells

The high-throughput assay systems, e.g., yeast three-hybrid systems, of the present disclosure can include one or more cells for the detection of a molecule, e.g., therapeutic molecule. In certain embodiments, one or more cells for use in the high-throughput systems of the present disclosure are genetically-engineered to include one or more nucleic acids and/or express one or more proteins, e.g., fusion proteins. In certain embodiments, a genetically-engineered cell for use in an assay of the present disclosure can (i) express a DNA-binding domain fusion protein (also referred to herein as the “first fusion protein”), (ii) express an activation domain fusion protein (also referred to herein as the “second fusion protein”) and/or (iii) include a reporter construct as disclosed below. In certain embodiments, the genetically-engineered cell can further include a heterodimeric ligand, e.g., a chemical inducer of dimerization (CID), as disclosed below.

In certain embodiments, the genetically-engineered cell for use in the disclosed assays can be a mammalian cell, a plant cell, a bacterial cell or a fungal cell. For example, but not by way of limitation, the genetically-engineered cell for use in an assay of the present disclosure can be a mammalian cell. In certain embodiments, the genetically-engineered cell for use in an assay of the present disclosure can be a plant cell. In certain embodiments, the genetically-engineered cell for use in an assay of the present disclosure can be a bacterial cell. In certain embodiments, the genetically-engineered cell for use in an assay of the present disclosure can be a fungal cell.

Any fungal strain can be used in the assays disclosed herein. In certain embodiments, the fungal cell can be a cell of Alternaria brasicicola, Arthrobotrys oligospora, Ashbya aceri, Ashbya gossypii, Aspergillus clavatus, Aspergillus flavus, Aspergillus fumigate, Aspergillus kawachii, Aspergillus nidulans, Aspergillus niger, Aspergillus oryzae, Aspergillus ruber, Aspergillus terreus, Baudoinia compniacensis, Beauveria bassiana, Botryosphaeria parva, Botrytis cinereal, Candida albicans, Candida dubliniensis, Candida glabrata, Candida guilliermondii, Candida lusitaniae, Candida parapsilosis, Candida tenuis, Candida tropicalis, Capronia coronate, Capronia epimyces, Chaetomium globosum, Chaetomium thermophilum, Chryphonectria parasitica, Claviceps purpurea, Coccidioides immitis, Colletotrichum gloeosporioides, Coniosporium apollinis, Dactylellina haptotyla, Debaryomyces hansenii, Endocarpon pusillum, Eremothecium cymbalariae, Fusarium oxysporum, Fusarium pseudograminearum, Gaeumannomyces graminis, Geotrichum candidum, Gibberella fujikuroi, Gibberella moniliformis, Gibberella zeae, Glarea lozoyensis, Grosmannia clavigera, Kazachstania Africana, Kazachstania naganishii, Kluyveromyces lactis, Kluyveromyces marxianus, Kluyveromyces waltii, Komagataella pastoris, Kuraishia capsulate, Lachancea kluyveri, Lachancea thermotolerans, Lodderomyces elongisporus, Magnaporthe oryzae, Magnaporthe poae, Marssonina brunnea, Metarhizium acridum, Metarhizium anisopliae, Mycosphaerella graminicola, Mycosphaerella pini, Nectria haematococca, Neosartorya fischeri, Neurospora crassa, Neurospora tetrasperma, Ogataea parapolymorpha, Ophiostoma piceae, Paracoccidioides lutzii, Penicillium chrysogenum, Penicillium digitatum, Penicillium oxalicum, Penicillium roqueforti, Phaeosphaeria nodorum, Pichia sorbitophila, Podospora anserine, Pseudogymnoascus destructans, Pyrenophora teres f teres, Pyrenophora tritici-repentis, Saccharomyces bayanus, Saccharomyces castellii, Saccharomyces cerevisiae, Saccharomyces dairenensis, Saccharomyces mikatae, Saccharomyces paradoxis, Scheffersomyces stipites, Schizosaccharomyces japonicus, Schizosaccharomyces octosporus, Schizosaccharomyces pombe, Sclerotinia borealis, Sclerotinia sclerotiorum, Sordaria macrospora, Sporothrix schenckii, Tetrapisispora blattae, Tetrapisispora phaffii, Thielavia heterothallica, Togninia minima, Torulaspora delbrueckii, Trichoderma atroviridis, Trichoderma jecorina, Trichoderma vixens, Tuber melanosporum, Vanderwaltozyma polyspora 1, Vanderwaltozyma polyspora 2, Verticillium alfalfae, Verticillium dahliae, Wickerhamomyces ciferrii, Yarrowia lipolytica, Zygosaccharomyces bailii or Zygosaccharomyces rouxii.

In certain embodiments, a genetically-engineered cell for use in an assay of the present disclosure can be a species of phylum Ascomycota. In certain embodiments, the species of the phylum Ascomycota is selected from Saccharomyces cerevisiae, Saccharomyces castellii, Saccharomyces var boulardii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum or Capronia coronate. In certain embodiments, the genetically-engineered cell is Saccharomyces cerevisiae.

Non-limiting examples of bacteria include Caulobacter crescentus, Rodhobacter sphaeroides, Pseudoalteromonas haloplanktis, Shewanella sp. strain Ac10, Pseudomonas jitiorescens, Pseudomonas aeruginosa, Halomonas elongata, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis

Lactobacillus plantarum, Lactobacillus casei, Lactobacillus reuteri, Lactobacillus gasseri and Escherichia coli. In certain embodiments, the bacteria cell is Escherichia coli.

Non-limiting examples of mammalian cells include the monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; FS4 cells; MCF-7 cells; 3T3 cells; U2SO cells; Chinese hamster ovary (CHO) cells; and myeloma cell lines such as Y0, NS0 and Sp2/0.

DNA-binding Domain Fusion Protein

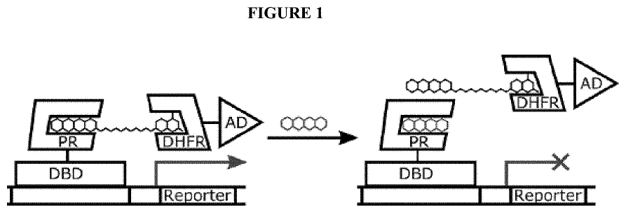

In certain embodiments, one or more cells, e.g., yeast cells, for use in the assays disclosed herein can express a first fusion protein. In certain embodiments, the first fusion protein includes a DNA-binding domain or a protein or fragment thereof that includes a DNA-binding domain (see FIG. 1). In certain embodiments, the DNA-binding domain is DNA-binding domain from a transcriptional activator or a transcriptional repressor. For example, and not by way of limitation, the DNA-binding domain can be the DNA-binding domain of LexA. In certain embodiments, the first fusion protein includes LexA or a DNA-binding domain thereof. In certain embodiments, the first fusion protein includes Gal4 or a DNA-binding domain thereof. In certain embodiments, the first fusion protein includes Ace1 or a DNA-binding domain thereof. Additional examples of DNA-binding domains are disclosed in Golemis et al. Current Protocols in Cell Biology 8(1):17.3.1-17.3.42 (2000), incorporated by reference herein in its entirety.

In certain embodiments, the first fusion protein can further include a ligand binding domain. For example, but not by way of limitation, the ligand binding domain is a protein or fragment thereof that binds to the molecule, e.g., therapeutic molecule, of interest (or a derivative or analogue of the therapeutic molecule of interest). In certain embodiments, the first fusion protein can include a DNA-binding domain coupled to a protein or fragment thereof that binds to the molecule, e.g., therapeutic molecule, of interest or a derivative or analogue of the therapeutic molecule of interest (see FIG. 1). For example, but not by way of limitation, the protein or fragment thereof that binds to the therapeutic molecule of interest can be a receptor for the molecule, e.g., therapeutic molecule, of interest. In certain embodiments, the protein or fragment thereof that binds to the molecule, e.g., therapeutic molecule, of interest, e.g., receptor, can bind tetracycline or an analogue or a derivative thereof, e.g., TAN-1612.

In certain embodiments, the protein that binds the molecule, e.g., therapeutic molecule, e.g., tetracycline or an analogue or a derivative thereof, can be a Tet repressor protein (TetR) or a variant thereof. Non-limiting examples of TetR proteins include TetR(A), TetR(B), TetR(C), TetR(D), TetR(E), TetR(G) and TetR(H) and variants thereof. In certain embodiments, the TetR can be TetR(B) or a variant thereof. In certain embodiments, the TetR is TetR(G) or a variant thereof. In certain embodiments, the TetR or variant thereof can be encoded by a nucleotide sequence that comprises the sequence of any one of SEQ ID NOs: 8-14. In certain embodiments, the TetR or variant thereof can be encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence comprising any one of SEQ ID NOs: 8-14. In certain embodiments, the TetR or variant thereof can be encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the sequence of SEQ ID NO: 9 or 13. In certain embodiments, the TetR or variant thereof can be encoded by a nucleotide sequence comprising any one of SEQ ID NOs: 8-14. For example, but not by way of limitation, the TetR protein is encoded by a nucleotide sequence that comprises the sequence of SEQ ID NO: 9 or 13.

In certain embodiments, the first fusion protein can be a LexA-TetR fusion protein. In certain embodiments, the LexA-TetR fusion protein can be encoded by a nucleotide sequence that comprises the sequence of any one of SEQ ID NOs: 1-7. In certain embodiments, the LexA-TetR fusion protein can be encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence comprising any one of SEQ ID NOs: 1-7. In certain embodiments, the LexA-TetR fusion protein can be encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the sequence of SEQ ID NO: 2 or 6. In certain embodiments, the LexA-TetR fusion protein can be encoded by a nucleotide sequence that comprises any one of SEQ ID NOs: 1-7. In certain embodiments, the LexA-TetR fusion protein can be encoded by a nucleotide sequence that comprises the sequence of SEQ ID NO: 2 or 6.

In certain embodiments, the first fusion protein can be expressed in a cell, e.g., a yeast cell, by introducing a nucleic acid that encodes the first fusion protein into the cell. For example, but not by way of limitation, the nucleic acid can include a nucleotide sequence that encodes the DNA-binding domain or a protein or fragment thereof that includes a DNA-binding domain. In certain embodiments, the nucleic acid can further include a nucleotide sequence that encodes the protein or fragment thereof that binds to the molecule, e.g., therapeutic molecule, of interest. In certain embodiments, the nucleic acid encoding the first fusion protein can comprise a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence comprising any one of SEQ ID NOs: 1-7. In certain embodiments, the nucleic acid encoding the first fusion protein comprises a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence comprising any one of SEQ ID NOs: 8-14. For example, but not by way of limitation, the nucleic acid encoding the first fusion protein comprises the nucleotide sequence of SEQ ID NO: 2 or 6. In certain embodiments, the nucleic acid encoding the first fusion protein comprises the nucleotide sequence of SEQ ID NO: 9 or 13.

Activation Domain Fusion Protein

In certain embodiments, one or more cells, e.g., yeast cells, of the assay system of the present disclosure can express a second fusion protein. In certain embodiments, the second fusion protein includes a domain that can regulate transcription, e.g., activate transcription. In certain embodiments, the second fusion protein includes a domain that can activate transcription e.g., an activation domain, or a protein or fragment thereof that includes a domain that can activate transcription, e.g., a protein or fragment thereof that includes an activation domain (see FIG. 1). In certain embodiments, the activation domain can be the activation domain of B42 (also referred to herein as the “transactivational domain of B42”). In certain embodiments, the first fusion protein includes B42 or an activation domain thereof. In certain embodiments, the first fusion protein includes VP16 or an activation domain thereof. In certain embodiments, the first fusion protein includes Gal4 or an activation domain thereof. In certain embodiments, the first fusion protein includes Ace1 or an activation domain thereof. Additional examples of activations domains are disclosed in Golemis et al. Current Protocols in Cell Biology 8(1):17.3.1-17.3.42 (2000), incorporated by reference herein in its entirety.

In certain embodiments, the second fusion protein can include a ligand binding domain. In certain embodiments, the ligand binding domain can be a protein or fragment thereof that binds to a small molecule. For example, but not by way of limitation, the small molecule can be methotrexate, FK506, dexamethasone, serotonin, progesterone, biotin, estradiol or derivatives or analogues thereof.

For example, but not by way of limitation, the second fusion protein can include a protein or fragment thereof that binds to methotrexate (see FIG. 1). For example, but not by way of limitation, the protein or fragment thereof that binds to methotrexate can be dihydrofolate reductase (DHFR) or a fragment thereof. In certain embodiments, the protein or fragment thereof that binds to methotrexate can be β-glycinamide ribonucleotide transformylase (GARFT) or a fragment thereof. In certain embodiments, the protein or fragment thereof that binds to methotrexate can be 5′-amino-4′-imidazolecarboxamide ribonucleotide transformylase (AICARFT) or a fragment thereof. In certain embodiments, the protein or fragment thereof that binds to methotrexate can be thymidylate synthetase (TYMS) or a fragment thereof. Additional non-limiting examples of proteins that bind methotrexate are disclosed in Cancer Manag. Res. 2:293-301(2010), which is incorporated by reference herein in its entirety.

Alternatively, the second fusion protein can include a protein or fragment thereof that binds to the small molecule FK506 or an analogue or derivative thereof. For example, but not by way of limitation, the protein or fragment thereof that binds to the small molecule FK506 can be FKBP12.

In certain embodiments, the second fusion protein can include a protein or fragment thereof that binds to dexamethasone. For example, but not by way of limitation, the protein or fragment thereof that binds to the small molecule dexamethasone can be the glucocorticoid receptor.

In certain embodiments, the second fusion protein can include a protein or fragment thereof that binds to serotonin. For example, but not by way of limitation, the protein or fragment thereof that binds to the small molecule serotonin can be the 5-HT receptor.

In certain embodiments, the second fusion protein can include a protein or fragment thereof that binds to progesterone. For example, but not by way of limitation, the protein or fragment thereof that binds to the small molecule progesterone can be the progesterone receptor.

In certain embodiments, the second fusion protein can include a protein or fragment thereof that binds to biotin. For example, but not by way of limitation, the protein or fragment thereof that binds to the small molecule biotin can be streptavidin.

In certain embodiments, the second fusion protein can include a protein or fragment thereof that binds to estradiol. For example, but not by way of limitation, the protein or fragment thereof that binds to the small molecule estradiol can be estrogen receptor.

Alternatively, the first fusion protein can include the protein or fragment thereof that binds to a small molecule and the second fusion protein can include the protein or fragment thereof that binds the molecule, e.g., therapeutic molecule, of interest. For example, but not by way of limitation, the first fusion protein can include (i) a DNA-binding domain and (ii) a protein or fragment thereof that binds to methotrexate, and second fusion protein can include (i) an activation domain and (ii) a protein or fragment thereof that binds to the molecule of interest.

In certain embodiments, the second fusion protein can be a DHFR-B42 fusion protein. In certain embodiments, the DHFR-B42 fusion protein can be encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the sequence of SEQ ID NO: 17. In certain embodiments, the second fusion protein can be encoded by a nucleotide sequence comprising the sequence of SEQ ID NO: 17.

In certain embodiments, the second fusion protein can be expressed in one or more cells, e.g., yeast cells, by introducing a nucleic acid that encodes the second fusion protein into the one or more cells. For example, but not by way of limitation, the nucleic acid can include a nucleotide sequence that encodes the activation domain or a protein or fragment thereof that includes an activation domain, e.g., B42. In certain embodiments, the nucleic acid can further include a nucleotide sequence that encodes the protein or fragment thereof that binds to methotrexate, e.g., DHFR. In certain embodiments, the second fusion protein can be a DHFR-B42 fusion protein. In certain embodiments, the nucleic acid encoding the second fusion protein, e.g., DHFR-B42 fusion protein, can comprise a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the sequence of SEQ ID NO: 17. In certain embodiments, the nucleic acid encoding the second fusion protein, e.g., DHFR-B42 fusion protein, can comprise a nucleotide sequence comprising the sequence of SEQ ID NO: 17.

Reporter Construct

In certain embodiments, one or more cells, e.g., yeast cells, of the assay systems of the present disclosure can include a nucleic acid that includes a DNA-binding site, e.g., that interacts with the first fusion protein comprising a DNA-binding domain (see FIG. 1). For example, but not by way of limitation, the DNA-binding site can be a binding site for the DNA-binding domain thereof of the first fusion protein. For example, but not by way of limitation, the DNA-binding site can be a binding site for LexA or a DNA-binding domain thereof.

In certain embodiments, the nucleic acid can further include a reporter gene (see FIG. 1). In certain embodiments, the reporter gene can encode a detectable reporter. In certain embodiments, the detectable reporter includes a label, e.g., a compound capable of emitting a detectable signal, including but not limited to fluorophores, chemiluminescent dyes, chromophores, enzymes, enzymes substrates, enzyme cofactors, enzyme inhibitors and the like. The term “fluorophore” refers to a substance or a portion thereof which is capable of exhibiting fluorescence in a detectable image. In certain embodiments, the detectable reporter can be a fluorescent protein. For example, but not by way of limitation, the fluorescent protein can be GFP, sfGFP, deGFP, eGFP, Venus, YFP, Cerulean, Citrine, CFP, eYFP, eCFP, RFP, mRFP, mCherry and mmCherry. In certain embodiments, the detectable reporter is mCherry. In certain embodiments, the detectable reporter is not a fluorescent protein. In certain embodiments, the detectable reporter can be a chromogenic reporter such as LacZ. In certain embodiments, the detectable reporter is not LacZ.

In certain embodiments, the nucleic acid includes the DNA-binding site for LexA and includes a nucleotide sequence encoding mCherry. In certain embodiments, the nucleic acid comprises a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the sequence of SEQ ID NO: 16. In certain embodiments, the nucleic acid including the DNA-binding site for LexA and the nucleotide sequence encoding mCherry comprises the sequence of SEQ ID NO: 16. In certain embodiments, the nucleic acid is present in a plasmid, e.g., pRS416, and comprises a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the sequence of SEQ ID NO: 15. In certain embodiments, the plasmid comprising a nucleic acid including the DNA-binding site for LexA and the nucleotide sequence encoding mCherry comprises the sequence of SEQ ID NO: 15.

In certain embodiments, the detectable reporter is expressed upon dimerization of the first fusion protein and the second fusion protein. In certain embodiments, the dimerization of the first fusion protein and the second fusion protein, e.g., by a chemical inducer of dimerization, induces proximity of the DNA-binding domain of the first fusion protein and the activation domain of the second fusion protein thereby resulting in transcription activation.

The detection of the reporter can be performed by various methods identifiable by those skilled in the art, such as in vitro methods: fluorescence, absorbance, mass spectrometry, flow cytometry, colorimetric, visual, UV, gas chromatography, liquid chromatography, Western blot and thin layer chromatography. In particular, a labeling signal can be quantitative or qualitatively detected with these techniques as will be understood by a skilled person. In certain embodiments, the term “labeling signal” as used herein indicates the signal emitted from the label that allows detection of the label, including but not limited to radioactivity, fluorescence, chemiluminescence, production of a compound in outcome of an enzymatic reaction (e.g., production of colored compounds) and the like. For example, but not by way of limitation, a fluorescent protein such as GFP can be detected with an excitation range of 485 and an emission range of 515, and mRFP can be detected with an excitation range of 580 and an emission range of 610. Other reportable molecular components do not require excitation to be detected; for example, colorimetric reportable molecular components can have a detectable color without fluorescent excitation.

In certain embodiments, the reporter gene encodes a reporter that cannot be detected by the naked eye (e.g., the change or appearance of the color cannot be detected by the naked eye), and/or a reporter whose detection requires instrumentation.

In certain embodiments, the reporter gene encodes a reporter that can be detected by the naked eye (e.g., the change or appearance of the color can be detected by the naked eye) and/or whose detection does not require instrumentation (e.g., reporters that are not conventionally used as research tools).

In certain embodiments, the reporter gene is a gene that regulates the growth of a cell, e.g., a selectable marker. In certain embodiments, the reporter gene is a conditionally-essential gene. In certain embodiments, a conditionally-essential gene can be a gene that is required to generate an essential amino acid, e.g., URA3, HIS3, LEU2 and TRP1. For example, the reporter gene encodes the uracil biosynthesis gene URA3. URA3 encodes orotidine-5′-phosphate decarboxylase (ODCase), which facilitates growth on media not supplemented with uracil or uridine. However, in the presence of 5-fluorototic acid (5-FOA), expression of URA3 converts 5-FOA into the toxic compound 5-fluorouracil causing cell death.

In certain embodiments, the genetically-engineered cell can include a heterodimeric ligand, e.g., a chemical inducer of dimerization (CID), as disclosed herein. For example, but not by way of limitation, the CID can comprise (i) a ligand for the therapeutic molecule-binding protein or fragment thereof of the first fusion protein and (ii) methotrexate.

In certain embodiments, a nucleic acid disclosed herein, e.g., a nucleic acid encoding a fusion protein, can include one or more one or more regulatory regions such as promoters, transcription factor binding sites, operators, activator binding sites, repressor binding sites, enhancers, protein-protein binding domains, RNA binding domains, DNA-binding domains, and other control elements known to a person skilled in the art. For example, but not by way of limitation, a nucleic acid is introduced into the yeast cell either as a construct or a plasmid in which it is operably linked to a promoter active in the yeast cell or such that it is inserted into the yeast cell genome at a location where it is operably linked to a suitable promoter. Non-limiting examples of suitable yeast promoters include, but are not limited to, constitutive promoters pTef1, pPgk1, pCyc1, pAdh1, pKex1, pTdh3, pTpi1, pPyk1 and pHxt7 and inducible promoters pGal1, pCup1, pMet15, pFig1 and pFus1. For example, but not by way of limitation, a nucleic acid encoding a fusion protein herein can include a constitutively active promoter, e.g., pAdh1. In certain embodiments, a nucleic acid can include an inducible promoter, e.g., pGal1.

In certain embodiments, the nucleic acid encoding a fusion protein, e.g., the LexA-TetR fusion protein, can include a constitutively active promoter, e.g., pAdh1. In certain embodiments, the nucleic acid encoding a fusion protein, e.g., the DHFR-B42 fusion protein, can include an inducible promoter, e.g., pGal1.

In certain embodiments, the nucleic acid comprising the reporter gene can include an inducible promoter, e.g., pGal1.

In certain embodiments, a cell for use in the disclosed assays can comprise one or more of: (a) a nucleic acid encoding a first fusion protein, e.g., a LexA-TetR fusion protein; (b) a nucleic acid encoding a second fusion protein, e.g., a DHFR-B42 fusion protein; and/or (c) a nucleic acid comprising a reporter gene, e.g., a nucleic acid comprises a DNA-binding site and a reporter gene, e.g., encoding mCherry. For example, but not by way of limitation, a cell for use in the disclosed assays can include (a); (a) and (b); (a) and (c); (a), (b) and (c); (b); (b) and (c); or (c). In certain embodiments, the first fusion protein and the second fusion protein can be encoded by a single nucleic acid.

In certain embodiments, nucleic acids of the present disclosure can be introduced into cells, e.g., yeast cells, using vectors, such as plasmid vectors and cell transformation techniques such as electroporation, heat shock and others known to those skilled in the art and described herein. In certain embodiments, the genetic molecular components are introduced into the cell to persist as a plasmid or integrate into the genome. For example, but not by way of limitation, the nucleic acid encoding the detectable reporter can be incorporated into the genome of the genetically-engineered cell. In certain embodiments, the cells can be engineered to chromosomally integrate a polynucleotide of one or more genetic molecular components described herein, using methods identifiable to skilled persons upon reading the present disclosure.

### III. Assays

The present disclosure further provides assays using the cells disclosed herein. In certain embodiments, the presently disclosed subject matter provides yeast three-hybrid assays using the genetically-engineered cells disclosed herein. The present disclosure further provides systems for performing the assays described herein.

In certain embodiments, the present disclosure provides assays for the detection of molecules, e.g., therapeutic molecules. In certain embodiments, the present disclosure provides assays for determining whether a molecule, e.g., therapeutic molecule, is produced or not produced by a cell. For example, but not by way of limitation, an assay disclosed herein can be used to detect the presence of a molecule, e.g., therapeutic molecule, produced by a genetically-engineered cell. In certain embodiments, an assay disclosed herein can be used to detect the presence of a molecule, e.g., therapeutic molecule, produced by a cell that is not genetically-engineered. In certain embodiments, an assay disclosed herein can be used to detect the presence of a molecule that is not a therapeutic produced by a genetically-engineered cell. In certain embodiments, an assay disclosed herein can be used to detect the presence of a molecule that is not a therapeutic produced by a cell that is not genetically-engineered. Non-limiting examples of molecules, e.g., therapeutic molecules, are provided below.

In certain embodiments, a method for detecting the presence of a therapeutic molecule can include providing a genetically-engineered cell that (i) expresses a DNA-binding domain fusion protein (i.e., a first fusion protein), (ii) expresses an activation domain fusion protein (i.e., a second fusion protein) and (iii) includes a nucleic acid that includes a DNA-binding site and a reporter-encoding sequence. For example, but not by way of limitation, the first fusion protein can be a LexA-TetR fusion protein. In certain embodiments, the second fusion protein can be a DHFR-B42 fusion protein. In certain embodiments, the nucleic acid can include a DNA-binding site for LexA and a nucleotide sequence that encodes LacZ, a fluorophore, e.g., mCherry, or is a conditionally-essential gene, e.g., URA3.

In certain embodiments, the assay can further include contacting the cell with a heterodimeric molecule that promotes the dimerization of the first fusion protein and the second fusion protein. In certain embodiments, the heterodimeric molecule can be a small molecule, a nucleic acid, a protein (e.g., a fusion protein) or a peptide. In certain embodiments, the heterodimeric molecule can comprise a ligand for the molecule-binding protein, e.g., therapeutic molecule-binding protein, or fragment thereof of the first fusion protein. For example, but not by way of limitation, the heterodimeric molecule can comprise a ligand (e.g., moiety) that binds to TetR of the first fusion protein. In certain embodiments, the heterodimeric molecule can comprise a ligand (e.g., moiety) that binds to the second fusion protein, e.g., DHFR of the second fusion protein. For example, but not by way of limitation, the heterodimeric molecule can comprise methotrexate, e.g., to bind DHFR of the second fusion protein. Alternatively, the heterodimeric molecule can comprise FK506, e.g., to bind FKBP12 of the second fusion protein.

In certain embodiments, the heterodimeric molecule is a chemical inducer of dimerization (CID). In certain embodiments, the CID can comprise a ligand (e.g., moiety) for the molecule-binding protein, e.g., therapeutic molecule-binding protein, or fragment thereof of the first fusion protein. For example, but not by way of limitation, the CID can comprise a ligand that binds to TetR of the first fusion protein. In certain embodiments, the CID can comprise a derivative of tetracycline as the ligand for TetR of the first fusion protein. In certain embodiments, the CID can comprise a ligand that binds to the second fusion protein, e.g., DHFR of the second fusion protein. For example, but not by way of limitation, the CID can also include methotrexate, e.g., for binding to DHFR of the second fusion protein. In certain embodiments, the CID can comprise methotrexate and a derivative of tetracycline, e.g., an 9-amido-tetracycline. In certain embodiments, the derivative of tetracycline is minocycline, e.g., 9-NH₂-minocycline. In certain embodiments, the derivative of tetracycline is doxycycline, e.g., 9-NH2-doxycycline. In certain embodiments, the CID can comprise methotrexate and minocycline. Alternatively, the CID can comprise FK506, e.g., for binding to FKBP12 of the second fusion protein. In certain embodiments, the CID can comprise FK506 and a derivative of tetracycline, e.g., minocycline.

In certain embodiments, the CID can be a heterodimeric minocycline-methotrexate molecule. In certain embodiments, the CID has the following chemical formula (also referred to herein as Min-Mtx):

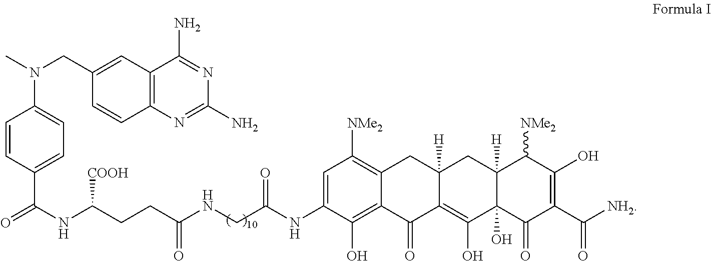

In certain embodiments, the CID of Formula I can be synthesized according to Scheme 1 (and as discussed in Example 1).

In certain embodiments, the assay can further include contacting the genetically-engineered cell with a sample. For example, but not by way of limitation, the sample can include the molecule, e.g., therapeutic molecule, to be detected. In certain embodiments, the sample does not include the molecule, e.g., therapeutic molecule. In certain embodiments, the sample can be the supernatant from the culture of a cell that expresses the molecule, e.g., therapeutic molecule, e.g., if the molecule is secreted. Alternatively, the cells producing or engineered to produce the molecule, e.g., therapeutic molecule, can be lysed and fractions of the lysed cells can be analyzed using the disclosed assays. In certain embodiments, the cell can be contacted with the CID and the sample simultaneously. Alternatively, the cell can be contacted with the CID followed by contact with the sample or the cell can be contacted with sample first followed by contact with the CID.

In certain embodiments, from about 1 to about 1×10⁷ cells can be used in an assay. For example, but not by way of limitation, from about 100 to about 1×10⁶ cells, from about 100 to about 1×10⁵ cells, from about 100 to about 1×10⁴ cells, from about 100 to about 1,000 cells, from about 1,000 to about 1×10⁷ cells, from about 1,000 to about 1×10⁶ cells, from about 1,000 to about 1×10⁵ cells or from about 1,000 to about 1×10⁴ cells.

In certain embodiments, the assay further includes detecting the reporter, e.g., expression of the reporter gene. In the presence of the CID, the first fusion protein and the second fusion protein dimerizes and induces expression of the reporter gene. In the presence of the molecule, e.g., therapeutic molecule, of interest, the molecule, e.g., therapeutic molecule, of interest will compete with CID for binding to TetR thereby blocking dimerization of the first and second fusion proteins and preventing expression of the reporter gene. As a result, the expression level of the reporter indicates the efficiency of the dimerization of the two fusion proteins. For example, but not by way of limitation, the greater amount of molecule, e.g., therapeutic molecule, present in the sample, the lower the expression level of the reporter. In certain embodiments, the lower the amount of molecule, e.g., therapeutic molecule, present in the sample, the higher the expression level of the reporter. In certain embodiments, reduced expression of the reporter gene as compared to a reference control indicates the presence of the therapeutic molecule in the sample. In certain embodiments, the reference control is the expression level of the reporter in the absence of the sample or in the presence of a sample that does not include the molecule, e.g., therapeutic molecule.

In certain embodiments, the reporter gene is a conditionally-essential gene, e.g., URA3. In certain embodiments, the assay can be performed in the presence of 5-FOA. For example, but not by way of limitation, in the presence of the CID, the first fusion protein and the second fusion protein dimerizes and induce expression of the reporter gene. Expression of URA3 results in the conversion of 5-FOA into a toxic compound resulting in cell death. In the presence of the molecule, e.g., therapeutic molecule, of interest, the molecule, e.g., therapeutic molecule, of interest will compete with CID for binding to TetR thereby blocking dimerization of the first and second fusion proteins and preventing expression of the reporter. As a result, the number of cells remaining after the assay indicates the efficiency of the dimerization of the two fusion proteins. For example, but not by way of limitation, the greater amount of molecule, e.g., therapeutic molecule, present in the sample, the greater the number of cells that remain after the assay. In certain embodiments, the lower the amount of molecule, e.g., therapeutic molecule, present in the sample, the lower the number of cells that remain after the assay. In certain embodiments, increased number of cells as compared to a reference control indicates the presence of the molecule, e.g., therapeutic molecule, in the sample. In certain embodiments, the reference control is the number of cells in the absence of the sample or in the presence of a sample that does not include the molecule, e.g., therapeutic molecule.

In certain embodiments, assays of the present disclosure can be used to assess the expression level of a molecule, e.g., therapeutic molecule, between different cells. For example, but not by way of limitation, the present disclosure provides assays for analyzing one or more cells that express a molecule, e.g., therapeutic molecule, and comparing the expression of the molecule by each of the cells to identify the cell that expresses the molecule at a higher level. For example, but not by way of limitation, expression of the reporter can be used to analyze the expression level of the one or more cells. In certain embodiments, the assay can include contacting two or more genetically-engineered cells individually with a CID. In certain embodiments, the two or more genetically-engineered cell each (i) express a DNA-binding domain fusion protein (i.e., a first fusion protein), (ii) express an activation domain fusion protein (i.e., a second fusion protein) and (iii) includes a nucleic acid that includes a DNA-binding site and a reporter-encoding sequence. The assay can further include contacting each of the genetically-engineered cells with a different sample, e.g., where each sample is from a different cell that produces a molecule of interest. The assay can further include detecting the reporter, e.g., expression of the reporter gene. In certain embodiments, the assay can further include comparing the expression of the reporter gene of each genetically-engineered cell to identify the genetically-engineered cell with the highest or lowest expression level, where the highest expression level indicates that the sample that has contacted that genetically-engineered cell produces the molecule at the lowest level and where the lowest expression level indicates that the sample that has contacted that genetically-engineered cell produces the molecule at the highest level. This assay can be used to identify high producing cells for further use in producing the molecule at a greater scale, e.g., large scale manufacturing.

In certain embodiments, assays of the present disclosure can be performed in assay plates or test tubes manufactured from polyethylene, polypropylene, polystyrene, and the like, including 96-well microtiter plates. In certain embodiments, an assay of the present disclosure is performed on a microtiter plate, e.g., a 96-well microtiter plate, to analyze several samples at one time.

The present disclosure further provides systems for performing the assays disclosed herein. For example, but not by way of limitation, a system of the present disclosure includes a genetically-engineered cell disclosed herein. For example, but not by way of limitation, the genetically-engineered cell can be a cell that (i) expresses a DNA-binding domain fusion protein (i.e., a first fusion protein), (ii) expresses an activation domain fusion protein (i.e., a second fusion protein) and (iii) includes a nucleic acid that includes a DNA-binding site and a reporter-encoding sequence. In certain embodiments, the first fusion protein can be a LexA-TetR fusion protein. In certain embodiments, the second fusion protein can be a DHFR-B42 fusion protein. In certain embodiments, the nucleic acid can include a DNA-binding site for LexA and a nucleotide sequence that encodes LacZ, a fluorophore, e.g., mCherry, or is a conditionally-essential gene, e.g., URA3. In certain embodiments, the system can further include a CID. In certain embodiments, the CID can comprise a ligand for the therapeutic molecule-binding protein or fragment thereof of the first fusion protein. In certain embodiments, the CID can comprise a ligand for the therapeutic molecule-binding protein or fragment thereof of the first fusion protein. In certain embodiments, the CID can comprise methotrexate and a derivative of tetracycline.

### IV. Molecules

The assays of the present disclosure can be used to detect a molecule, e.g., the presence and/or level of a molecule in a sample. In certain embodiments, the present disclosure provides assays for detecting a therapeutic molecule, e.g., the presence and/or level of a therapeutic molecule in a sample. For example, but not by way of limitation, the assays, e.g., yeast three-hybrid systems, of the present disclosure can be used to detect the generation of a molecule, e.g., therapeutic molecule, produced by a cell, e.g., a cell genetically-engineered to synthesize the therapeutic molecule.

In certain embodiments, the molecule can be a small molecule. In certain embodiments, the molecule can be a protein or a fragment thereof. In certain embodiments, the molecule can be a peptide.

In certain embodiments, the therapeutic molecule can be a small therapeutic molecule. In certain embodiments, the therapeutic molecule can be a protein or a fragment thereof. In certain embodiments, the therapeutic molecule can be a peptide.

Small Molecule Therapeutics

In certain embodiments, the molecule, e.g., therapeutic molecule, can be a small therapeutic molecule. In certain embodiments, genetically-engineered or non genetically-engineered cells, e.g., modified strain of yeasts, that produce and/or secretes a small therapeutic molecule by engineered biosynthesis can be analyzed by the assays disclosed herein. Small therapeutic molecules are molecules with a low molecular weight, generally less than 900 Daltons. Non-limiting examples of small molecules are disclosed in, e.g., Orange Book: Approved Drug Products with Therapeutic Equivalence Evaluations. Rockville, Md.: U.S. Dept. of Health and Human Services, Food and Drug Administration, Center for Drug Evaluation and Research, Office of Pharmaceutical Science, Office of Generic Drugs, 2019. Internet resource, which is incorporated herein in its entirety and analogs thereof.

In certain embodiments, the small molecule therapeutic is one or more of an analgesic drug, an antibiotic drug, an anticoagulant drug, an antidepressant drug, an anticancer drug, an antineoplastic drug, a cytotoxic drug, a chemotherapy drug, an antiepileptic drug, an antipsychotic drug, an antiviral drug, a sedative drug, an antidiabetic drug, a cardiovascular drug, an immunomodulatory agent, an antiinflammatory drug, an antifungal, an antimicrobial or an antithrombotic.

In certain embodiments, the small molecule therapeutic can be an inhibitor of a kinase, e.g., a tyrosine or a seine/threonine kinase. Non-limiting examples of such inhibitors include imanitib, gefitinib, erlotinib, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, vandetanib and perifosine. Additional examples of small molecule therapeutics are provided in Chahrour et al., Mini-Reviews in Medicinal Chemistry 12 (2012), which is incorporated by reference herein in its entirety.

In certain embodiments, the small molecule therapeutic can be thebaine, hydrocodone, Δ9-tetrahydrocannabinolic acid, simvastatin, lovastatin, artemisinin, penicillin, taxols, e.g., paclitaxel or docetaxel, erythromycin, tetracycline, chlortetracycline, oxytetracycline, demeclocycline, meclocycline, metacycline, doxycycline, minocycline, tigecycline, omadacycline, sarecycline, eravacycline, anhydrotetracycline, TAN-1612, viridicatumtoxin and analogues or derivatives thereof.

In certain embodiments, the small molecule therapeutic is tetracycline or a derivative thereof. For example, but not by way of limitation, the tetracycline derivative can be TAN-1612. In certain embodiments, the tetracycline derivative is doxycycline. In certain embodiments, the tetracycline derivative is a 9-amido-tetracycline. In certain embodiments, the tetracycline derivative is chlortetracycline. In certain embodiments, the tetracycline derivative is oxytetracycline. In certain embodiments, the tetracycline derivative is demeclocycline. In certain embodiments, the tetracycline derivative is meclocycline. In certain embodiments, the tetracycline derivative is metacycline. In certain embodiments, the tetracycline derivative is doxycycline. In certain embodiments, the tetracycline derivative is minocycline. In certain embodiments, the tetracycline derivative is tigecycline. In certain embodiments, the tetracycline derivative is omadacycline. In certain embodiments, the tetracycline derivative is sarecycline. In certain embodiments, the tetracycline derivative is eravacycline. In certain embodiments, the tetracycline derivative is anhydrotetracycline. In certain embodiments, the tetracycline derivative is 4-de(dimethylamino)-anhydrotetracycline. In certain embodiments, the tetracycline derivative is viridicatumtoxin. In certain embodiments, the tetracycline derivative is an analogue or derivative of the above.

Protein and Peptide Therapeutics

In certain embodiments, the molecule, e.g., therapeutic molecule, can be a peptide or a protein or fragment thereof.

In certain embodiments, the molecule, e.g., therapeutic molecule, can be a protein or a fragment thereof. In certain embodiments, the protein, e.g., protein therapeutic can have a molecular weight of at least about 15-100 kD, e.g., closer to about 15 kD. In certain embodiments, the protein therapeutic can include at least about 50, about 60, about 70, about 80, about 90, about 100, about 200, about 300, about 400, about 500 amino acids, about 1,000 amino acids, about 1,500 amino acids, about 2,000 amino acids, about 2,500 amino acids, about 3,000 amino acids, about 35,000 amino acids or about 40,000 amino acids. Non-limiting examples of protein therapeutics include all proteins, and, in general proteins that contain one or more disulfide bonds, including multi-chain polypeptides comprising one or more inter- and/or intrachain disulfide bonds. In certain embodiments, the protein therapeutics, e.g., antibodies, can include other post-translation modifications including, but not limited to, glycosylation and lipidation. See, e.g., Prabakaran et al., WIREs Syst Biol Med (2012), which is incorporated herein by reference in its entirety.

In certain embodiments, the molecule, e.g., therapeutic molecule, can be a peptide. In certain embodiments, the peptides, e.g., therapeutic peptides, can be composed of about 3-50 amino acid residues. In certain embodiments, the 3-50 amino acid residues can be continuous within a larger polypeptide or protein or can be a group of 3-50 residues that are discontinuous in a primary sequence of a larger polypeptide or protein but that are spatially near in three-dimensional space. In certain embodiments, the therapeutic peptide can be a part of a peptide, a part of a full protein or polypeptide and can be released from that protein or polypeptide by proteolytic treatment or can remain part of the protein or polypeptide.

In certain embodiments, the peptide, e.g., therapeutic peptide, can have a length of 3 residues or more, a length of 4 residues or more, a length of 5 residues or more, 6 residues or more, 7, residues or more, 8 residues or more, 9 residues or more, 10 residues or more, 11 residues or more, 12 residues or more, 13 residues or more, 14 residues or more, 15 residues or more, 16 residues or more, 17 residues or more, 18 residues or more, 19 residues or more, 20 residues or more, 21 residues or more, 22 residues or more, 23 residues or more, 24 residues or more, 25 residues or more, 26 residues or more, 27 residues or more, 28 residues or more, 29 residues or more, 30 residues or more, 31 residues or more, 32 residues or more, 33 residues or more, 34 residues or more, 35 residues or more, 36 residues or more, 37 residues or more, 38 residues or more, 39 residues or more, 40 residues or more, 41 residues or more, 42 residues or more, 43 residues or more, 44 residues or more, 45 residues or more, 46 residues or more, 47 residues or more, 48 residues or more, 49 residues or more or 50 residues or more. In certain embodiments, the peptide 1 has a length of 3-50 residues, 5-50 residues, 3-45 residues, 5-45 residues, 3-40 residues, 5-40 residues, 3-35 residues, 5-35 residues, 3-30 residues, 5-30 residues, 3-25 residues, 5-25 residues, 3-20 residues, 5-20 residues, 3-15 residues, 5-15 residues, 3-10 residues, 3-10 residues, 5-10 residues, 10-15 residues, 15-20 residues, 20-25 residues, 25-30 residues, 30-35 residues, 35-40 residues, 40-45 residues or 45-50 residues. In certain embodiments, the therapeutic peptide has a length of about 5 to about 30 residues.

In certain embodiments, the therapeutic peptide has a length of 9 residues. In certain embodiments, the therapeutic peptide has a length of 10 residues. In certain embodiments, the therapeutic peptide has a length of 11 residues. In certain embodiments, the therapeutic peptide has a length of 12 residues. In certain embodiments, the therapeutic peptide has a length of 13 residues. In certain embodiments, the therapeutic peptide has a length of 14 residues. In certain embodiments, the therapeutic peptide has a length of 15 residues. In certain embodiments, the therapeutic peptide has a length of 16 residues. In certain embodiments, the therapeutic peptide has a length of 17 residues. In certain embodiments, the therapeutic peptide has a length of 18 residues. In certain embodiments, the therapeutic peptide has a length of 99 residues. In certain embodiments, the therapeutic peptide has a length of 20 residues. In certain embodiments, the therapeutic peptide has a length of 21 residues. In certain embodiments, the therapeutic peptide has a length of 22 residues. In certain embodiments, the therapeutic peptide has a length of 23 residues. In certain embodiments, the therapeutic peptide has a length of 24 residues. In certain embodiments, the therapeutic peptide has a length of 25 residues. In certain embodiments, the therapeutic peptide has a length of 26 residues. In certain embodiments, the therapeutic peptide has a length of 27 residues. In certain embodiments, the therapeutic peptide has a length of 28 residues. In certain embodiments, the therapeutic peptide has a length of 29 residues. In certain embodiments, the peptide, e.g., therapeutic peptide, has a length of 30 residues. In certain embodiments, the therapeutic peptide has a length of 31 residues. In certain embodiments, the therapeutic peptide has a length of 32 residues. In certain embodiments, the therapeutic peptide has a length of 33 residues. In certain embodiments, the therapeutic peptide has a length of 34 residues. In certain embodiments, the therapeutic peptide has a length of 35 residues. In certain embodiments, the therapeutic peptide has a length of 36 residues. In certain embodiments, the therapeutic peptide has a length of 37 residues. In certain embodiments, the therapeutic peptide has a length of 38 residues. In certain embodiments, the therapeutic peptide has a length of 39 residues. In certain embodiments, the therapeutic peptide has a length of 40 residues. In certain embodiments, the therapeutic peptide has a length of 41 residues. In certain embodiments, the therapeutic peptide has a length of 42 residues. In certain embodiments, the therapeutic peptide has a length of 43 residues. In certain embodiments, the therapeutic peptide has a length of 44 residues. In certain embodiments, the therapeutic peptide has a length of 45 residues. In certain embodiments, the therapeutic peptide has a length of 46 residues. In certain embodiments, the therapeutic peptide has a length of 47 residues. In certain embodiments, the therapeutic peptide has a length of 48 residues. In certain embodiments, the therapeutic peptide has a length of 49 residues. In certain embodiments, the therapeutic peptide has a length of 50 residues.

In certain embodiments, the protein or peptide therapeutic is one or more of an analgesic drug, an antibiotic drug, an anticoagulant drug, an antidepressant drug, an anticancer drug, an antineoplastic drug, a cytotoxic drug, a chemotherapy drug, an antiepileptic drug, an antipsychotic drug, an antiviral drug, a sedative drug, an antidiabetic drug, a cardiovascular drug, an immunomodulatory agent, an antiinflammatory drug, an antifungal, an antimicrobial or an antithrombotic.

In certain embodiments, the therapeutic protein can be insulin, Interferon-a n1, Interferon-a n3, Sargramostim (127 residue glycoprotein), Interferon Alfa-2a, Recombinant, Tumor necrosis factor (TNF-alpha) binding antibody (chimeric IgG1), Recombinant type-I Interferon alpha 2b, anti-CD20 antibodies, interferon alfa-2b, anti-IL-6R antibodies, IL-1R accessory protein, Tenectaplase, Urate-oxidase enzyme or beta-glucocerebrosidase.

In certain embodiments, the peptide and/or protein therapeutic can be an antibody or an antigen binding fragment thereof.

In certain embodiments, the peptide and/or protein therapeutic can be selected from the group consisting of hirudin, bivalirudin, abatacept, abciximab, adalimumab, aflibercept, agalsidase beta, albiglutide, aldesleukin, alemtuzumab, alglucosidase alfa, alirocumab, alpha-1-proteinase inhibitor, alteplase, anakinra, ancestim, anistreplase, antihaemophilic factor, antithrombin iii human, antithymocyte globulin, arcitumomab, asfotase alfa, asparaginase, asparaginase Erwinia chrysanthemi, atezolizumab, basiliximab, becaplermin, belatacept, belimumab, beractant, bevacizumab, bevalirudin, blinatumomab, botulinum toxin type a, botulinum toxin type b, brentuximab vedotin, buserelin, cl esterase inhibitor (human), canakinumab, capromab, certolizumab pegol, cetuximab, choriogonadotropin alfa, coagulation factor ix, coagulation factor viia, coagulation factor xiii a-subunit (recombinant), collagenase, corticotropin, cosyntropin, daclizumab, daptomycin, daratumumab, darbepoetin alfa, defibrotide, denileukin, denosumab, desirudin, digoxin immune fab (ovine), dornase alfa, dulaglutide, eculizumab, efalizumab, elosulfase alfa, elotuzumab, enfuvirtide, epifabitide, epoetin alfa, epoetin zeta, etanercept, evolocumab, exenatide, fibrinogen concentrate (human), filgrastim, filgrastim-sndz, follitropin beta, galsulfase, gastric intrinsic factor, glatirmer acetate, glucagon recombinant, glucarpidase, golimumab, gramicidin d, hepatitis b immune globulin, human calcitonin, human Clostridium tetani toxoid immune globulin, human rabies virus immune globulin, human serum albumin, human rho(d) immune globulin, hyaluronidase, hyaluronidase (human ecombinant), ibritumomab, idarucizumab, idursulfase, imiglucerase, immune globulin human, infliximab, insulin aspart, insulin beef, insulin degludec, insulin detemir, insulin glargine, insulin glulisine, insulin isophane, insulin lispro, insulin pork, insulin regular, insulin, porcine, interferon alfa-n3, interferon alfa-2b, interferon alfacon-1, interferon alpha 2a recombinant, interferon alpha n1, interferon beta-1a, interferon beta-1b, interferon gamma 1b, intravenous mmunoglobin, ipilimumab, ixekizumab, laronidase, lepirudin, leuprolide, liraglutide, lucinactant, lutropin alfa, mecasermin, menotropins, mepolizumab, methoxy polyethylene glycol-epoetin beta, metreleptin, muromonab, natalizumab, natural alpha interferon, necitumumab, nesiritide, nivolumab, obiltoxaximab, obinutuzumab, ocriplasmin, ofatumumab, omalizumab, oprelvekin, oxytocin, palifermin, palivizumab, pancrelipase, panitumumab, pegademase bovine, pegaptanib, pegaspargase, pegfilgrastim, peginterferon, peginterferon alfa 2b, peginterferon beta-1a, peginterferon beta-1a, pegvisomant, pembrolizumab, pertuzumab, poractant alfa, pramlintide, preotact, prothrombin complex concentrate, ramucirumab, ranibizumab, rasburicase, raxibacumab, reteplase, rilonacept, rituximab, romiplastin, sacrosidase, salmon calcitonin, sargramostim, sebelipase alfa, secretin, serum albumin, serum albumin iodinated, siltuximab, simoctocog alfa, streptokinase, taliglucerase alfa, teduglutide, teicoplanin, tenecteplase, teriparatide, tesamorelin, thymalfasin, thyroglobulin, thyrotropin alfa, tocilizumab, tositumomab, trastuzumab, tuberculin purified protein derivative, turoctocog alfa, urofollitropin, ustekinumab, vasopressin, vedolizumab and velaglucerase alfa. In certain embodiments, the peptide and/or protein comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence of any one of the proteins or peptides disclosed above.

Therapeutic Molecule Producing Cells

The assays, e.g., yeast three-hybrid systems, of the present disclosure can be used to detect the generation of a molecule, e.g., therapeutic molecule, produced by a cell, e.g., a genetically-engineered cell. For example, but not by way of limitation, the cell, e.g., genetically-engineered cell, producing the therapeutic molecule can be a mammalian cell, a plant cell, a bacterial cell or a fungal cell. In certain embodiments, the cell can be a plant cell, e.g., a genetically-engineered plant cell. In certain embodiments, the cell producing the molecule is not genetically-engineered.

In certain embodiments, the cell can be a bacterial cell, e.g., a genetically-engineered bacterial cell. Non-limiting examples of bacteria include Caulobacter crescentus, Rodhobacter sphaeroides, Pseudoalteromonas haloplanktis, Shewanella sp. strain Ac10, Pseudomonas fluorescens, Pseudomonas aeruginosa, Halomonas elongata, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, Lactobacillus gasseri, and Escherichia coli. In certain embodiments, the bacteria cell is Escherichia coli.

In certain embodiments, the cell can be a mammalian cell, e.g., a genetically-engineered mammalian cell. Non-limiting examples of mammalian cells include the monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; FS4 cells; MCF-7 cells; 3T3 cells; U2SO cells; Chinese hamster ovary (CHO) cells; and myeloma cell lines such as Y0, NS0 and Sp2/0.

In certain embodiments, the cell producing the therapeutic molecule can be a fungal cell, e.g., a genetically-engineered fungal cell. For example, but not by way of limitation, the cell can be a cell of Alternaria brasicicola, Arthrobotrys oligospora, Ashbya aceri, Ashbya gossypii, Aspergillus clavatus, Aspergillus flavus, Aspergillus fumigate, Aspergillus kawachii, Aspergillus nidulans, Aspergillus niger, Aspergillus oryzae, Aspergillus ruber, Aspergillus terreus, Baudoinia compniacensis, Beauveria bassiana, Botryosphaeria parva, Botrytis cinereal, Candida albicans, Candida dubliniensis, Candida glabrata, Candida guilliermondii, Candida lusitaniae, Candida parapsilosis, Candida tenuis, Candida tropicalis, Capronia coronate, Capronia epimyces, Chaetomium globosum, Chaetomium thermophilum, Chryphonectria parasitica, Claviceps purpurea, Coccidioides immitis, Colletotrichum gloeosporioides, Coniosporium apollinis, Dactylellina haptotyla, Debaryomyces hansenii, Endocarpon pusillum, Eremothecium cymbalariae, Fusarium oxysporum, Fusarium pseudograminearum, Gaeumannomyces graminis, Geotrichum candidum, Gibberella fujikuroi, Gibberella moniliformis, Gibberella zeae, Glarea lozoyensis, Grosmannia clavigera, Kazachstania Africana, Kazachstania naganishii, Kluyveromyces lactis, Kluyveromyces marxianus, Kluyveromyces waltii, Komagataella pastoris, Kuraishia capsulate, Lachancea kluyveri, Lachancea thermotolerans, Lodderomyces elongisporus, Magnaporthe oryzae, Magnaporthe poae, Marssonina brunnea, Metarhizium acridum, Metarhizium anisopliae, Mycosphaerella graminicola, Mycosphaerella pini, Nectria haematococca, Neosartorya fischeri, Neurospora crassa, Neurospora tetrasperma, Ogataea parapolymorpha, Ophiostoma piceae, Paracoccidioides lutzii, Penicillium chrysogenum, Penicillium digitatum, Penicillium oxalicum, Penicillium roqueforti, Phaeosphaeria nodorum, Pichia sorbitophila, Podospora anserine, Pseudogymnoascus destructans, Pyrenophora teres f teres, Pyrenophora tritici-repentis, Saccharomyces bayanus, Saccharomyces castellii, Saccharomyces cerevisiae, Saccharomyces dairenensis, Saccharomyces mikatae, Saccharomyces paradoxis, Scheffersomyces stipites, Schizosaccharomyces japonicus, Schizosaccharomyces octosporus, Schizosaccharomyces pombe, Sclerotinia borealis, Sclerotinia sclerotiorum, Sordaria macrospora, Sporothrix schenckii, Tetrapisispora blattae, Tetrapisispora phaffii, Thielavia heterothallica, Togninia minima, Torulaspora delbrueckii, Trichoderma atroviridis, Trichoderma jecorina, Trichoderma vixens, Tuber melanosporum, Vanderwaltozyma polyspora 1, Vanderwaltozyma polyspora 2, Verticillium alfalfae, Verticillium dahliae, Wickerhamomyces ciferrii, Yarrowia lipolytica, Zygosaccharomyces bailii, Zygosaccharomyces rouxii and combinations thereof.

In certain embodiments, the cell, e.g., genetically-engineered cell, producing the therapeutic molecule can be a species of phylum Ascomycota. In certain embodiments, the species of the phylum Ascomycota is selected from Saccharomyces cerevisiae, Saccharomyces castellii, Saccharomyces var boulardii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, Capronia coronate and combinations thereof. In certain embodiments, the genetically-engineered cell producing the therapeutic molecule is Saccharomyces cerevisiae.

In certain embodiments, the cell, e.g., genetically-engineered cell, can produce and/or secrete one molecule, e.g., therapeutic molecule, described herein. In certain embodiments, the cell, e.g., genetically-engineered cell, can produce and/or secrete more than one molecule, e.g., therapeutic molecule, e.g., two therapeutic molecules, three therapeutic molecules, four therapeutic molecules or five or more therapeutic molecules. In certain embodiments, a multi-cell system can be used for the generation of pharmaceuticals that require the assembly of multiple components in a coordinated manner, where each cell is configured to produce a component of a pharmaceutical. Non-limiting examples of multi-cell systems, e.g., a scalable G-protein coupled receptor (GPCR)-ligand intercellular signaling system, are disclosed in PCT/US2020/030795, the contents of which are incorporated by reference herein. In certain embodiments, a multi-cell system can be used for the generation of multiple different molecules. In certain embodiments, a multi-cell system can be used for the generation of, e.g., about 2, about 3, about 4, about 5, about 6, about 7, about 8, about 9 or about 10 different therapeutic molecules or precursors (and/or intermediates) to generate a therapeutic molecule.

### V. Kits

The present disclosure further provides kits for performing an assay, e.g., a yeast-three hybrid assay, of the present disclosure.

In certain embodiments, a kit of the present disclosure can include a nucleic acid comprising (i) a DNA-binding site and (ii) a reporter gene. In certain embodiments, the reporter gene is a conditionally-essential gene, e.g., URA3, or encodes LacZ or a fluorescent protein. In certain embodiments, the DNA-binding site is a binding site for LexA. In certain embodiments, the nucleic acid comprises a nucleotide sequence that is at least about 90% homologous to a sequence of any one of SEQ ID NOs: 15-16.

In certain embodiments, a kit of the present disclosure can include a nucleic acid encoding a first fusion protein comprising (i) a DNA-binding domain that binds to the DNA-binding site and (ii) a protein or fragment thereof that binds to a therapeutic molecule. In certain embodiments, the DNA-binding domain of the first fusion protein is LexA. In certain embodiments, the protein or fragment thereof that binds to a therapeutic molecule of the first fusion protein is a tetracycline receptor (TetR), e.g., TetR(A), TetR(B), TetR(C), TetR(D), TetR(E), TetR(G) or TetR(H) or a variant thereof. In certain embodiments, the first fusion protein is encoded by a nucleotide sequence that is at least about 90% homologous to a sequence of any one of SEQ ID NOs: 1-7. In certain embodiments, the protein or fragment thereof that binds to a therapeutic molecule is encoded by a nucleotide sequence that is at least about 90% homologous to a sequence of any one of SEQ ID NOs: 8-14.

In certain embodiments, a kit of the present disclosure can include a nucleic acid encoding a second fusion protein comprising (i) an activation domain and (ii) a protein or fragment thereof that binds to methotrexate. In certain embodiments, the activation domain of the second fusion protein is B42. In certain embodiments, the protein or fragment thereof that binds to methotrexate is dihydrofolate reductase (DHFR). In certain embodiments, the second fusion protein is encoded by a nucleotide sequence that is at least about 90% homologous to the sequence of SEQ ID NO: 17.

In certain embodiments, a kit of the present disclosure can include a chemical inducer of dimerization (CID), wherein the CID comprises (i) a ligand that binds to the protein or fragment thereof that binds to the therapeutic molecule of the first fusion protein and (ii) methotrexate. In certain embodiments, the CID can include methotrexate and a derivative of tetracycline. For example, but not by way of limitation, the CID can comprise minocycline and methotrexate.

In certain embodiments, a kit of the present disclosure can include a cell, e.g., a genetically-engineered cell comprising one or more of the nucleic acids disclosed herein. For example, but not by way of limitation, a kit of the present disclosure can include a genetically-engineered cell comprising one or more of: (a) a nucleic acid encoding a first fusion protein, e.g., a LexA-TetR fusion protein; (b) a nucleic acid encoding a second fusion protein, e.g., a DHFR-B42 fusion protein; and/or (c) a nucleic acid comprising a reporter gene, e.g., a nucleic acid comprises a DNA-binding site and a reporter gene, e.g., encoding mCherry. For example, but not by way of limitation, the genetically-engineered cell can include (a); (a) and (b); (a) and (c); (a), (b) and (c); (b); (b) and (c); or (c). In certain embodiments, the first fusion protein and the second fusion protein can be encoded by a single nucleic acid. Alternatively, the kit can include one or more cells, e.g., yeast cells, in one container and can further include one or more disclosed nucleic acids in a second container. In certain embodiments, the genetically-engineered cell can further include a CID as described herein.

In certain embodiments, a kit of the present disclosure can include a cell, e.g., a genetically-engineered cell, disclosed herein. For example, but not by way of limitation, a kit can include a genetically-engineered cell that (i) expresses a DNA-binding domain fusion protein (i.e., a first fusion protein), (ii) expresses an activation domain fusion protein (i.e., a second fusion protein) and (iii) includes a nucleic acid that includes a DNA-binding site and a reporter-encoding sequence. In certain embodiments, the first fusion protein can be a LexA-TetR fusion protein. In certain embodiments, the second fusion protein can be a DHFR-B42 fusion protein. In certain embodiments, the nucleic acid can include a DNA-binding site for LexA and a nucleotide sequence that encodes LacZ, a fluorophore, e.g., mCherry, or is a conditionally-essential gene, e.g., URA3. In certain embodiments, the kit can further include a CID. In certain embodiments, the CID can comprise a ligand for the therapeutic molecule-binding protein or fragment thereof of the first fusion protein. In certain embodiments, the CID can comprise a ligand for the therapeutic molecule-binding protein or fragment thereof of the first fusion protein. In certain embodiments, the CID can comprise methotrexate and a derivative of tetracycline.

### EXAMPLES

The following examples are put forth so as to provide those of ordinary skill in the art with a complete disclosure and description of how to make and use the presently disclosed subject matter and are not intended to limit the scope of what the inventors regard as their presently disclosed subject matter. It is understood that various other implementations and embodiments can be practiced, given the general description provided herein.

### Example 1. Methods

The following methods were used in the Examples disclosed herein.

General methods. Absorption and fluorescence spectra were recorded on an Infinite-M200 fluorescent spectrometer. DNA sequences were purchased from IDT. Polymerases, restriction enzymes, and Gibson Assembly mix were purchased from New England Biolabs. Sanger sequencing was performed by Genewiz. Yeast strains were grown at 30° C., and the shaker setting was 200 rpm, unless otherwise indicated. Yeast transformations were done using the lithium acetate method.¹⁹ Plasmids were cloned and amplified using Gibson Assembly and cloning strain C3040 (New England Biolabs).²⁰ Unless otherwise indicated, yeast strains were grown on synthetic minimal media lacking histidine and/or uracil and/or tryptophan and/or leucine, as indicated by the abbreviation HUTL-.²¹ Tables 1 and 2 list the strains and plasmids used in these examples. Yeast strain patches were obtained from glycerol stocks by streaking on an agar plate of synthetic medium lacking the appropriate amino acid markers, incubating at 30° C. for 3 days, patching single colonies onto a fresh agar plate, and incubating at 30° C. overnight. Unless otherwise indicated, when 96-well plates were placed in the yeast shaker, 95% of the interface between the plate and its cover was wrapped with laboratory film (Parafilm, Bemis PM999). All Y3H assays were performed with sterile components and three biological replicates.

TABLE 1
Strains used in these examples.

| Strain | Genotype | Source/Reference |
|---|---|---|
| FY250 | MATα ura3-52 trp1 63 his3 200 leu2 1 gal | L. P. Guarente¹ |
| LW2635 | MATα trp1Δ63 his3Δ200 8lexAop-Spo13-URA3 | /^{[2]} |
| | leu2Δ1 Gal+ | |
| BJ5464-NpgA | MATα ura3-52 his3-Δ200 leu2-Δ1 | Y. Tang²/^{[3]} |
| | trp1 pep4::HIS3 δ:: pADH2-npgA-tADH2 prb1Δ1.6R can1 GAL | |
| V506 | FY250 pMW106, pMW3(GSG)2rGR2, pMW2eDHFR | /^{[4]} |
| PBA-5 | FY250 pMW106, pMW2eDHFR | This study |
| HL-260-1 | FY250 pMW106, pMW103TetR(A), pMW2eDHFR | This study |
| PBA-8 | FY250 pMW106, pMW103TetR(B), pMW2eDHFR | This study |
| HL-260-2 | FY250 pMW106, pMW103TetR(C), pMW2eDHFR | This study |
| HL-260-3 | FY250 pMW106, pMW103TetR(D), pMW2eDHFR | This study |
| HL-260-4 | FY250 pMW106, pMW103TetR(E), pMW2eDHFR | This study |
| HL-260-5 | FY250 pMW106, pMW103TetR(G), pMW2eDHFR | This study |
| HL-260-6 | FY250 pMW106, pMW103TetR(H), pMW2eDHFR | This study |
| HL-260-7 | FY250 pMW106, pMW103(empty), pMW2eDHFR | This study |
| PBA-6 | LW2635 PBA-Gib5 | This study |
| PBA-14 | LW2635 PBA-Gib5, pMW2eDHFR | This study |
| PBA-3 | FY250 pMW2eDHFR | This study |
| HL-294-7 | FY250 pMW2eDHFR HL-174-2 | This study |
| HL-2-4-1 | FY250 pMW2eDHFR pMW103TetR(B), HL-174-2 | This study |
| EH-3-54-4 | BJ5464-NpgA pYR291, pYR342 | This study |
| EH-3-54-2 | BJ5464-NpgA pRS426, pYR342 | This study |

^{[2]}Harton, M. D., Wingler, L. M., and Cornish, V. W. (2013) Transcriptional regulation improves the throughput of three-hybrid counter selections in Saccharomyces cerevisiae, Biotechnol J 8, 1485-1491;
^{[3]}Ma, S. M., Li, J. W. H., Choi, J. W., Zhou, H., Lee, K. K. M., Moorthie, V. A., Xie, X., Kealey, J. T., Da Silva, N. A., Vederas, J. C., and Tang, Y. (2009) Complete Reconstitution of a Highly Reducing Iterative Polyketide Synthase, Science 326, 589-592;
^{[4]}Abida, W. M., Carter, B. T., Althoff, E. A., Lin, H., and Cornish, V. W. (2002) Receptor-dependence of the transcription read-out in a small-molecule three-hybrid system, ChemBioChem 3, 887-895.

All chemical reactions were carried out under an argon atmosphere with anhydrous solvents unless otherwise indicated. Analytical high-pressure liquid chromatography (HPLC) was performed with a C-18 column, 2.6 μ, 250 mm×4.6 mm, with the eluent given in parentheses. Unless stated otherwise, reactions were monitored using analytical HPLC with a separation gradient of 10-90% MeCN in 0.1% (v/v) TFA aqueous solution over 20 min. Preparative HPLC was carried out with a C-18 5μ column, 250 mm×10 mm, with the eluent given in parentheses. NMR spectra were obtained using Bruker 400 or 500 MHz instruments, as indicated. Unless stated otherwise, mass spectroscopy measurements were performed on an Advion CMS mass spectrometer equipped with atmospheric pressure chemical ionization (APCI) and electrospray ionization (ESI) sources. TAN-1612 quantification was performed using a phenyl-hexyl 1.7 μ 100 mm×3 mm column, on a Waters SQD2 quadrupole mass spectrometer equipped with a UPC2 SFC inlet, a photodiode array (PDA) UV—vis detector, and a dual ESI/APCI probe. Unless stated otherwise, all reagents, salts, and solvents were purchased from commercial sources and used without further purification.

TABLE 2
Plasmids used in these examples.

| Plasmid | Description | Source/Reference |
|---|---|---|
| pMW2eDHFR | pGal1-eDHFR-B42, trp marker, 2 μ | /^{[4]} |
| pMW106 | LacZ under control of 8 tandem LexA operators, Ura marker | /^{[4, 5]} |
| pMW103 | pADH1-LexA (empty), His marker, 2 μ | R. Brent³/^{[5]} |
| pMW112 | LacZ under control of 8 tandem LexA operators, Ura marker | R. Brent/^{[5, 6]} |
| HL-249-1 | pADH1-LexA-TetR(A)-tADH1 on pMW103 | This study |
| PBA-Gfb5 | pADH1-LexA-TetR(B)-tADH1 on pMW103 | This study |
| HL-249-2 | pADH1-LexA-TetR(C)-tADH1 on pMW103 | This study |
| HL-249-3 | pADH1-LexA-TetR(D)-tADH1 on pMW103 | This study |
| HL-249-4 | pADH1-LexA-TetR(E)-tADH1 on pMW103 | This study |
| HL-249-5 | pADH1-LexA-TetR(G)-tADH1 on pMW103 | This study |
| HL-249-6 | pADH1-LexA-TetR(H)-tADH1 on pMW103 | This study |
| pRS416 | Shuttle vector (empty), uracil marker, CEN | ATCC87521 |
| HL-174-2 | 8LexAOp-minpGal1-mCherry-tCyc1, Ura marker, CEN | This study |
| pYR291 | AdaA expression in S. cerevisiae, uracil marker, 2 μ | Y. Tang⁴/^{[7]} |
| pYR342 | AdaB-D expression in S. cerevisiae, tryptophan marker, 2 μ | Y. Tang/^{[7]} |
| pRS426 | Shuttle vector (empty), uracil marker, 2 μ | ATCC77107 |
| pET21d-TetR | TetR(B) with C-term Histidine tag | A. Davidson⁵/^{[8]} |
| yEpGAP-Cherry | mCherry, uracil marker, 2 μ | Neta Dean⁶/^{[9]} |

^{[4]}Abida, W. M., Carter, B. T., Althoff, E. A., Lin, H., and Cornish, V. W. (2002) Receptor-dependence of the transcription read-out in a small-molecule three-hybrid system, ChemBioChem 3, 887-895;
^{[5]}Watson, M. A., Buckholz, R., and Weiner, M. P. (1996) Vectors encoding alternative antibiotic resistance for use in the yeast two-hybrid system, BioTechniques 21, 255-259;
^{[6]}Golemis, E. A., Serebriiskii, I., Finley, R. L., Kolonin, M. G., Gyuris, J., and Brent, R. (2008) Interaction Trap/Two-Hybrid System to Identify Interacting Proteins, Curr. Protoc. Cell Biol. 53, 17.13.11-17.13.35;
^{[7]}Li, Y. R., Chooi, Y. H., Sheng, Y. W., Valentine, J. S., and Tang, Y. (2011) Comparative Characterization of Fungal Anthracenone and Naphthacenedione Biosynthetic Pathways Reveals an alpha-Hydroxylation-Dependent Claisen-like Cyclization Catalyzed by a Dimanganese Thioesterase, J. Am. Chem. Soc. 133, 15773-15785;
^{[8]}Reichheld, S. E., and Davidson, A. R. (2006) Two-way interdomain signal transduction in tetracycline repressor, J. Mol. Biol. 361, 382-389;
^{[9]}Keppler-Ross, S., Noffz, C., and Dean, N. (2008) A New Purple Fluorescent Color Marker for Genetic Studies in Saccharomyces cerevisiae and Candida albicans, Genetics 179, 705-710.

Synthesis of the Min-Mtx CID (Scheme 1). Procedure A. To 9-NH2-minocycline (100 mg, 0.2 mmol), Boc-11-aminoundecanoic acid (89 mg, 0.3 mmol), EDC (75 mg, 0.4 mmol), and HOBt (60 mg, 0.4) were added DMF (1 mL) and Et3N (139 μL, 1.0 mmol), and the reaction was mixed at rt for 6 h. The reaction progress was monitored by RP-HPLC. Aqueous NaHCO₃(0.05 M)²² was then added, and the aqueous phase was extracted three times with CHCl₃. The combined organic fraction was extracted with brine, and the combined brine extracts were extracted twice with CHCl₃. The combined organic fraction was dried with Na₂SO₄, and the solvent was removed under reduced pressure. The crude product was purified in two batches by preparative RP-HPLC (30-35% MeCN in 99.9%:0.1% H2O/TFA, 45 min) to afford compound 3 (53.4 mg, 36%) as a yellow solid. 1 H NMR (400 MHz, MeOD-d4): δ 8.60 (s, 1H), 4.11 (s, 1H), 3.24 (dd, J=15.4, 4.2, 1H), 3.14 (s, 6H), 3.08 (m, 3H), 3.02 (s, 6H), 2.50 (t, J=7.4, 2H), 2.44 (t, J=14.3, 1H), 2.24 (m, 1H), 1.72 (m, 1H), 1.62 (m, 1H), 1.43 (s, 9H), 1.42 (m, 4H), 1.38 (m, 2H), 1.32 (m, 10H). MS (APCI+): m/z calcd for C₃₉H₅₈N₅O₁₀—, 756.42; found, 756.4 [M+H]+. MS (APCI−): m/z calcd for C₃₉H₅₆N₅O₁₀—, 754.40; found, 754.3 [M−H]−.

Procedures B-D. To compound 3 (27.3 mg, 0.036 mmol) were added DCM (0.5 mL) and TFA (0.5 mL), and the reaction was mixed at rt for 12 min. The solvent was removed under reduced pressure. PhMe was added, and the solvent was again removed under reduced pressure. PhMe addition and solvent removal was repeated once more. Methotrexate-α-OtBu (27.5 mg, 0.054 mmol)^{23′24} and PyBOP (37.5 mg, 0.072 mmol) were added, followed by DIPEA (11.5 mg, 0.09 mmol) in DMF (1 mL), and the reaction was mixed at rt. After 22 h, EtOAc and aqueous NaHCO₃ were added, and the aqueous phase was extracted twice with EtOAc. The combined organic fraction was extracted with brine, and the brine extract was filtered. The solid residue was redissolved in MeOH, and the solvent was removed under reduced pressure. The crude product was purified in two batches by preparative RP-HPLC (20-30% MeCN in 99.9%:0.1% H2O/TFA, 50 min). Following solvent removal under reduced pressure, TFA (2 mL) was added and the reaction was stirred at rt for 90 min. The reaction progress was monitored by RP-HPLC. The solvent was removed under reduced pressure to afford compound 1 (4.1 mg, 10%) as a yellow solid. ¹H NMR (500 MHz, MeOD-d4): δ 8.64 (s, 1H), 8.25 (s, 1H), 7.76 (d, J=8.8, 2H), 6.86 (d, J=8.8, 2H), 4.92 (s, 2H), 4.54 (dd, J=8.8, 4.5, 1H), 4.08 (s, 1H), 3.27 (s, 3H), 3.11 (m, 2H), 3.00 (s, 6H), 2.83 (s, 3H), 2.80, (s, 3H), 2.47 (m, 2H), 2.41-2.05 (m, 6H), 1.70-1.59 (m, 4H), 1.48-1.28 (m, 16H). MS (ESI+): m/z calcd for C₅₄H₇₁N₁₃O₁₂₂+, 1093.54; found, 546.9 [M+H]2+.

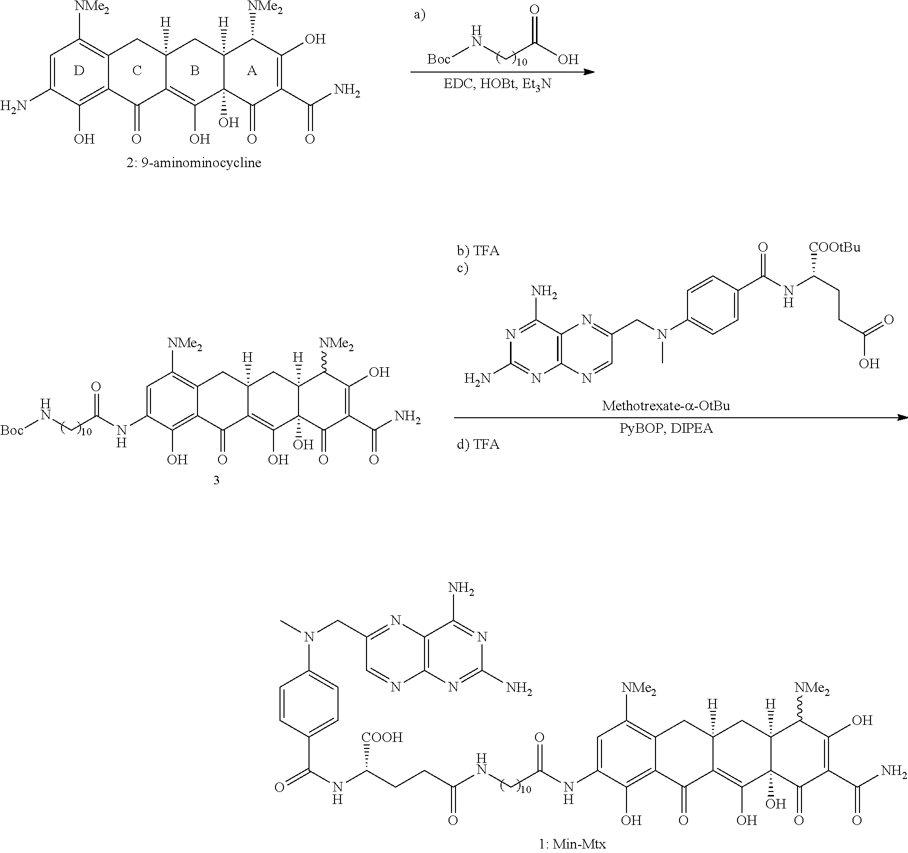

Scheme 1. Synthesis of the Chemical Inducer of Dimerization (CID) Min-Mtx. Reagents and conditions: (a) Boc-11-aminoundecanoic acid (1.5 equiv), EDC (2 equiv), HOBt (2 equiv), Et3N (5 equiv), DMF, 22° C., 6 h, 36%; (b) TFA/DCM (1:1), 22° C., 12 min; (c) methotrexate-α-OtBu (1.5 equiv),^{23,24} PYBOP (2 equiv), DIPEA (2.5 equiv), DMF, 22° C., 22 h; (d) TFA, 22° C., 1.5 h, 10% over three procedures. EDC=N-(3-(dimethylamino)propyl)-N′-ethylcarbodiimide hydrochloride, HOBt=1-hydroxybenzotriazole, TFA=trifluoroacetic acid, PyBOP=(benzotriazol-1-yloxy)tripyrrolidinophosphonium hexafluorophosphate, DIPEA=N-ethyldiisopropylamine.

Y3H Strain Construction. LexA-TetR Plasmids. Plasmids PBA-Gib5 and HL-249-1 through HL-249-6 encoding LexA-TetR protein fusions for the various TetR classes were cloned by Gibson Assembly of the appropriate TetR sequence with pMW103 digested with restriction enzymes BamHI-HF and EcoRI-HF (Table 3). All TetR sequences with the exception of B) were obtained as IDT gBlocks. TetR(B) sequence was amplified from pET21d-TetR, courtesy of A. Davidson.^{25,26}

TABLE 3
Sequences of the various TetR classes within
the pMW103 backbone. Sequences for the TetR
classes are shown decapitalized within the
context of LexA-DBD (partial, capitalized)
in the 5′ end and the tADH1 terminator
at the 3′ end (partial, capitalized).

| TetR Class | Sequence |
|---|---|
| TetR(A)- | GCTGCGCGTCAGCGGGATGTCGATG |
| HL-249-1 | AAAGATATCGGCATTATGGATGGTG |
| | ACTTGCTGGCAGTGCATAAAACTCA |
| | GGATGTACGTAACGGTCAGGTCGTT |
| | GTCGCACGTATTGATGACGAGGTTA |
| | CCGTTAAGCGCCTGAAAAAACAGGG |
| | CAATAAAGTCGAACTGTTGCCAGAA |
| | AATAGCGAGTTTAAACCAATTGTCG |
| | TAGATCTTCGTCAGCAGAGCTTCAC |
| | CATTGAAGGGCTGGCGGTTGGGGTT |
| | ATTCGCAACGGCGACTGGCTGGAAT |
| | TCatgacaaagttgcagccgaatac |
| | agtgatccgtgccgccctggacctg |
| | ttgaacgaggtcggcgtagacggtc |
| | tgacgacacgcaaactggcggaacg |
| | gttgggggttcagcagccggcgctt |
| | tactggcacttcaggaacaagcggg |
| | cgctgctcgacgcactggccgaagc |
| | catgctggcggagaatcatacgcat |
| | tcggtgccgagagccgacgacgact |
| | ggcgctcatttctgatcgggaatgc |
| | ccgcagcttcaggcaggcgctgctc |
| | gcctaccgcgatggcgcgcgcatcc |
| | atgccggcacgcgaccgggcgcacc |
| | gcagatggaaacggccgacgcgcag |
| | cttcgcttcctctgcgaggcgggtt |
| | tttcggccggggacgccgtcaatgc |
| | gctgatgacaatcagctacttcact |
| | gttggggccgtgcttgaggagcagg |
| | ccggcgacagcgatgccggcgagcg |
| | cggcggcaccgttgaacaggctccg |
| | ctctcgccgctgttgcgggccgcga |
| | tagacgccttcgacgaagccggtcc |
| | ggacgcagcgttcgagcagggactc |
| | gcggtgattgtcgatggattggcga |
| | aaaggaggctcgttgtcaggaacgt |
| | tgaaggaccgagaaagggtgacgat |
| | tagGATCCGTCGACCATGGCGGCCG |
| | CTCGAGTCGACCTGCAGCCAAGCTA |
| | ATTCCGGGCGAATTTCTTATGATTT |
| | ATGATTTTTATTATTAAATAAGTTA |
| | TAAAAAAAATAAGTGTATACAAATT |
| | TTAAAGTGACTCTTAGGTTTTAAAA |
| | CGAAAATTCTTATTCTTGAGTAACT |
| | (SEQ ID NO: 1). |
| | The decapitalized sequence |
| | Is SEQ ID NO: 8. |
| TetR(B)- | GCTGCGCGTCAGCGGGATGTCGATG |
| PBA-Gib5 | AAAGATATCGGCATTATGGATGGTG |
| | ACTTGCTGGCAGTGCATAAAACTCA |
| | GGATGTACGTAACGGTCAGGTCGTT |
| | GTCGCACGTATTGATGACGAGGTTA |
| | CCGTTAAGCGCCTGAAAAAACAGGG |
| | CAATAAAGTCGAACTGTTGCCAGAA |
| | AATAGCGAGTTTAAACCAATTGTCG |
| | TAGATCTTCGTCAGCAGAGCTTCAC |
| | CATTGAAGGGCTGGCGGTTGGGGTT |
| | ATTCGCAACGGCGACTGGCTGGAAT |
| | TCatgtctagattagataaaagtaa |
| | agtgattaacagcgcattagagctg |
| | cttaatgaggtcggaatcgaaggtt |
| | taacaacccgtaaactcgcccagaa |
| | gctaggtgtagagcagcctacattg |
| | tattggcatgtaaaaaataagcggg |
| | ctttgctcgacgccttagccattga |
| | gatgttagataggcaccatactcac |
| | ttttgccctttagaaggggaaagct |
| | ggcaagattttttacgtaataacgc |
| | taaaagttttagatgtgctttacta |
| | agtcatcgcgatggagcaaaagtac |
| | atttaggtacacggcctacagaaaa |
| | acagtatgaaactctcgaaaatcaa |
| | ttagcctttttatgccaacaaggtt |
| | tttcactagagaatgcattatatgc |
| | actcagcgctgtggggcattttact |
| | ttaggttgcgtattggaagatcaag |
| | agcatcaagtcgctaaagaagaaag |
| | ggaaacacctactactgatagtatg |
| | ccgccattattacgacaagctatcg |
| | aattatttgatcaccaaggtgcaga |
| | gccagccttcttattcggccttgaa |
| | ttgatcatatgcggattagaaaaac |
| | aacttaaatgtgaaagtgggtctta |
| | gGATCCGTCGACCATGGCGGCCGCT |
| | CGAGTCGACCTGCAGCCAAGCTAAT |
| | TCCGGGCGAATTTCTTATGATTTAT |
| | GATTTTTATTATTAAATAAGTTATA |
| | AAAAAAATAAGTGTATACAAATTTT |
| | AAAGTGACTCTTAGGTTTTAAAACG |
| | AAAATTCTTATTCTTGAGTAACT |
| | (SEQ ID NO: 2). The |
| | decapitalized sequence |
| | is SEQ ID NO: 9. |
| TetR(C)- | GCTGCGCGTCAGCGGGATGTCGATG |
| HL-249-2 | AAAGATATCGGCATTATGGATGGTG |
| | ACTTGCTGGCAGTGCATAAAACTCA |
| | GGATGTACGTAACGGTCAGGTCGTT |
| | GTCGCACGTATTGATGACGAGGTTA |
| | CCGTTAAGCGCCTGAAAAAACAGGG |
| | CAATAAAGTCGAACTGTTGCCAGAA |
| | AATAGCGAGTTTAAACCAATTGTCG |
| | TAGATCTTCGTCAGCAGAGCTTCAC |
| | CATTGAAGGGCTGGCGGTTGGGGTT |
| | ATTCGCAACGGCGACTGGCTGGAAT |
| | TCatgaacaagctccaacgcgaggc |
| | cgtgatccgaaccgcgctcgaactg |
| | cttaacgacgtgggcatggaaggtc |
| | taacgacgcgccGactggctgagcg |
| | cctcggggtgcaacagccagcgctc |
| | tactggcatttcaagaacaagcgtg |
| | cgttgctcgacgcacttgccgaagc |
| | catgctgacgataaatcacacgcat |
| | tcgacgccaagggatgacgacgact |
| | ggcgttcgttcctgaagggcaatgc |
| | atgcagttttcgacgggcgttgctc |
| | gcttatcgcgatggcgcgcgtattc |
| | atgccgggacgcggccagccgcgcc |
| | gcagatggaaaaagccgacgcgcag |
| | cttcgcttcctttgcgatgctggct |
| | tttcggcaggtgacgcgacctatgc |
| | gttgatggcaatcagctacttcacc |
| | gtcggcgctgttcttgagcagcaag |
| | ctagcgaggcagacgccgaggagcg |
| | gggcgaagatcagttgaccacctca |
| | gcgtctacgatgccggcgcgcctac |
| | agagcgcgatgaaaatcgtctacga |
| | aggcggtccggacgcggcattcgag |
| | cgaggcctggctctcatcatcggcg |
| | gtcttgaaaaaatgaggctcactac |
| | gaacgacattgaggtgctgaagaat |
| | gttgacgaatagGATCCGTCGACCA |
| | TGGCGGCCGCTCGAGTCGACCTGCA |
| | GCCAAGCTAATTCCGGGCGAATTTC |
| | TTATGATTTATGATTTTTATTATTA |
| | AATAAGTTATAAAAAAAATAAGTGT |
| | ATACAAATTTTAAAGTGACTCTTAG |
| | GTTTTAAAACGAAAATTCTTATTCT |
| | TGAGTAACT (SEQ ID NO: 3). |
| | The decapitalized sequence |
| | is SEQ ID NO: 10. |
| TetR(D)- | GCTGCGCGTCAGCGGGATGTCGATG |
| HL-249-3 | AAAGATATCGGCATTATGGATGGTG |
| | ACTTGCTGGCAGTGCATAAAACTCA |
| | GGATGTACGTAACGGTCAGGTCGTT |
| | GTCGCACGTATTGATGACGAGGTTA |
| | CCGTTAAGCGCCTGAAAAAACAGGG |
| | CAATAAAGTCGAACTGTTGCCAGAA |
| | AATAGCGAGTTTAAACCAATTGTCG |
| | TAGATCTTCGTCAGCAGAGCTTCAC |
| | CATTGAAGGGCTGGCGGTTGGGGTT |
| | ATTCGCAACGGCGACTGGCTGGAAT |
| | TCatggcacggctgaacagagaatc |
| | ggttattgatgcggcactggaactg |
| | ctgaatgagacagggattgacgggc |
| | tgacgacccgcaagctggcgcagaa |
| | gctgggaatagaacagccgacactt |
| | tactggcatgtgaaaaataaacggg |
| | cgttactggatgcgctggcggtgga |
| | gatcctggcgcgtcatcatgattat |
| | tcactgcctgcggcgggggaatcct |
| | ggcagtcatttctgcgcaataatgc |
| | aatgagtttccgccgggcgctgctg |
| | cgttaccgtgacggggcaaaagtgc |
| | acctcggcacccgccctgatgaaaa |
| | acagtatgatacggtggaaacccag |
| | ttacgctttatgacagaaaacggct |
| | tttcactgcgcgacgggttatatgc |
| | gatttcagcggtcagtcattttacc |
| | cttggtgccgtactggagcagcagg |
| | agcatactgccgccctgaccgaccg |
| | ccctgcagcaccggacgaaaacctg |
| | ccgccgctattgcgggaagcgctgc |
| | agattatggacagtgatgatggtga |
| | gcaggcctttctgcatggcctggag |
| | agcctgatccgggggtttgaggtgc |
| | agcttacggcactgttgcaaatagt |
| | cggtggtgataaacttatcatcccc |
| | ttttgctagGATCCGTCGACCATGG |
| | CGGCCGCTCGAGTCGACCTGCAGCC |
| | AAGCTAATTCCGGGCGAATTTCTTA |
| | TGATTTATGATTTTTATTATTAAAT |
| | AAGTTATAAAAAAAATAAGTGTAT |
| | ACAAATTTTAAAGTGACTCTTAGG |
| | TTTTAAAACGAAAATTCTTATTCTT |
| | GAGTAACT |
| | (SEQ ID NO: 4). |
| | The decapitalized sequence |
| | is SEQ ID NO: 11. |
| TetR(E)- | GCTGCGCGTCAGCGGGATGTCGATG |
| HL-249-4 | AAAGATATCGGCATTATGGATGGTG |
| | ACTTGCTGGCAGTGCATAAAACTCA |
| | GGATGTACGTAACGGTCAGGTCGTT |
| | GTCGCACGTATTGATGACGAGGTTA |
| | CCGTTAAGCGCCTGAAAAAACAGGG |
| | CAATAAAGTCGAACTGTTGCCAGAA |
| | AATAGCGAGTTTAAACCAATTGTCG |
| | TAGATCTTCGTCAGCAGAGCTTCAC |
| | CATTGAAGGGCTGGCGGTTGGGGTT |
| | ATTCGCAACGGCGACTGGCTGGAAT |
| | TCatggcacgactaagcttggacga |
| | cgtaatttcaatggcgctcaccctg |
| | ctggacagcgaagggctagagggct |
| | tgactacgcgtaagctggcgcagtc |
| | cctaaaaattgagcaaccgactctg |
| | tattggcacgtgcgcaacaagcaga |
| | ctcttatgaacatgctttcagaggc |
| | aatactggcgaagcatcacacccgt |
| | tcagcaccgttaccgactgagagtt |
| | ggcagcagtttctccaggaaaatgc |
| | tctgagtttccgtaaagcattactg |
| | gtccatcgtgatggagcccgattgc |
| | atatagggacctctcctacgccccc |
| | ccagtttgaacaagcagaggcgcaa |
| | ctacgctgtctatgcgatgcagggt |
| | tttcggtcgaggaggctcttttcat |
| | tctgcaatctatcagccattttacg |
| | ttgggtgcagtattagaggagcaag |
| | caacaaaccagatagaaaataatca |
| | tgtgatagacgctgcaccaccatta |
| | ttacaagaggcatttaatattcagg |
| | cgagaacctctgctgaaatggcctt |
| | ccatttcgggctgaaatcattaata |
| | tttggattttctgcacagttagatg |
| | aaaaaaagcatacacccattgagga |
| | tggtaataaatagGATCCGTCGACC |
| | ATGGCGGCCGCTCGAGTCGACCTGC |
| | AGCCAAGCTAATTCCGGGCGAATTT |
| | CTTATGATTTATGATTTTTATTATT |
| | AAATAAGTTATAAAAAAAATAAGTG |
| | TATACAAATTTTAAAGTGACTCTTA |
| | GGTTTTAAAACGAAAATTCTTATTC |
| | TTGAGTAACT (SEQ ID NO: 5). |
| | The decapitalized |
| | sequence is SEQ ID NO: 12. |
| TetR(G)- | GCTGCGCGTCAGCGGGATGTCGATG |
| HL-249-5 | AAAGATATCGGCATTATGGATGGTG |
| | ACTTGCTGGCAGTGCATAAAACTCA |
| | GGATGTACGTAACGGTCAGGTCGTT |
| | GTCGCACGTATTGATGACGAGGTTA |
| | CCGTTAAGCGCCTGAAAAAACAGGG |
| | CAATAAAGTCGAACTGTTGCCAGAA |
| | AATAGCGAGTTTAAACCAATTGTCG |
| | TAGATCTTCGTCAGCAGAGCTTCAC |
| | CATTGAAGGGCTGGCGGTTGGGGTT |
| | ATTCGCAACGGCGACTGGCTGGAAT |
| | TCatgaccaaactggacaagggcac |
| | cgtgatcgcggcggcgctagagctg |
| | ttgaacgaggttggcatggacagcc |
| | tgacgacgcggaagctcgctgaacg |
| | cctcaaggttcagcagcctgcgctt |
| | tactggcatttccagaacaagcgag |
| | cgctgcttgatgcgctcgccgaggc |
| | gatgctggcggaacgccatacccgc |
| | tcgctacccgaagagaatgaggact |
| | ggcgggtgttcctgaaagagaatgc |
| | cctgagcttcagaacggcgttgctc |
| | tcttatcgggacggcgcgcgtatcc |
| | atgccggcactcgaccgacagaacc |
| | gaattttggcaccgccgagacgcaa |
| | atacgctttctctgcgcggagggct |
| | tttgtccgaagcgcgccgtttgggc |
| | gctccgggcggtcagtcactatgtg |
| | gtcggttccgttctcgagcagcagg |
| | catctgatgccgatgagagagttcc |
| | ggacaggccagatgtgtccgagcaa |
| | gcaccgtcgtccttcctgcacgatc |
| | tgtttcacgagttggaaacagacgg |
| | catggatgctgcgttcaacttcgga |
| | ctcgacagcctcatcgctggtttcg |
| | agcggctgcgttcatctacaacaga |
| | ttagGATCCGTCGACCATGGCGGCC |
| | GCTCGAGTCGACCTGCAGCCAAGCT |
| | AATTCCGGGCGAATTTCTTATGATT |
| | TATGATTTTTATTATTAAATAAGTT |
| | ATAAAAAAAATAAGTGTATACAAAT |
| | TTTAAAGTGACTCTTAGGTTTTAA |
| | AACGAAAATTCTTATTCTTGAGTAA |
| | CT |
| | (SEQ ID NO: 6). |
| | The decapitalized sequence |
| | is SEQ ID NO: 13. |
| TetR(H)- | GCTGCGCGTCAGCGGGATGTCGATGA |
| HL-249-6 | AAGATATCGGCATTATGGATGGTGA |
| | CTTGCTGGCAGTGCATAAAACTCAG |
| | GATGTACGTAACGGTCAGGTCGTTG |
| | TCGCACGTATTGATGACGAGGTTAC |
| | CGTTAAGCGCCTGAAAAAACAGGGC |
| | AATAAAGTCGAACTGTTGCCAGAAA |
| | ATAGCGAGTTTAAACCAATTGTCGT |
| | AGATCTTCGTCAGCAGAGCTTCACC |
| | ATTGAAGGGCTGGCGGTTGGGGTTA |
| | TTCGCAACGGCGACTGGCTGGAATT |
| | Catggcaaagctagataaagaacaa |
| | gttattgatgatgcgttgattttac |
| | ttaatgaagttggtattgaaggatt |
| | aacaacgcgtaacgtggcgcaaaaa |
| | ataggtgtggaacaacccacattgt |
| | attggcatgtaaaaaataaacgcgc |
| | tttgttagatgcattagcagaaact |
| | attttgcaaaagcaccatcatcatg |
| | ttttgccattgccgaatgaaacatg |
| | gcaggactttttgcgaaataacgcg |
| | aaaagcttccgccaagccttattaa |
| | tgtatcgtgatggtggcaaaattca |
| | tgcgggaacacgcccctctgaaagt |
| | caatttgagacatcagaacagcaac |
| | tacagtttttgtgtgatgctgggtt |
| | tagtctatctcaagccgtgtatgca |
| | ttaagctctattgcgcattttacat |
| | taggctccgtactggaaactcaaga |
| | gcatcaagaaagccaaaaagagcgt |
| | gaaaaagtagagacggatactgttg |
| | cctatccgccattattaacccaagc |
| | cgttgcaattatggatagtgataat |
| | ggtgatgctgcatttttgtttgtcc |
| | ttgatgtgatgatctctggacttga |
| | aacagtattaaagagcgctaaatag |
| | GATCCGTCGACCATGGCGGCCGCTC |
| | GAGTCGACCTGCAGCCAAGCTAATT |
| | CCGGGCGAATTTCTTATGATTTATG |
| | ATTTTTATTATTAAATAAGTTATAA |
| | AAAAAATAAGTGTATACAAATTTTA |
| | AAGTGACTCTTAGGTTTTAAAACGA |
| | AAATTCTTATTCTTGAGTAACT |
| | (SEQ ID NO: 7). |
| | The decapitalized sequence |
| | is SEQ ID NO: 14. |

LacZ Assay Strain Construction. V506 was grown overnight in UT-media.²⁷ The culture was then diluted 104-105× with UT- and plated on UT-plates. Single colonies were streaked on HTU- and UT-plates and a streak growing on UT- but not on HTU-, indicating the loss of the pMW3-(GSG)2rGR2 plasmid, was glycerol stocked as PBA5. This strain was further used for the transformation of the LexA-TetR plasmids to generate LacZ assay strains PBA8 and HL-260-1 through HL-260-7.

Growth Assay Strain Construction. Yeast strain LW2635²⁸ was transformed with plasmid PBA-Gib5 to generate strain PBA6. PBA6 was transformed with pMW2eDHFR27 to generate the Y3H growth assay strain PBA14.

mCherry Reporter Plasmid Reporter plasmid HL-174-2 encoding mCherry under the transcriptional control of LexA operators was cloned by Gibson Assembly of an mCherry sequence amplified from yEpGAP-Cherry29 and an 8LexAmin-pGal1 sequence amplified from pMW11214 into pRS416 digested with SacI and XhoI.

TABLE 4
Sequence of 8LexAOp-minpGal1-mCherry-tCyc1 in
the context of pRS416-HL-174-2. The sequence
of 8-LexAOp-minpGal1-mCherry is
decapitalized and the context is
capitalized.

| | |
|---|---|
| HL-174-2 | ATACGACTCACTATAGGGCGAATTG |
| | GGTACCGGCCGCAAATTAAAGCCTT |
| | CGAGCGTCCCAAAACCTTCTCAAGC |
| | AAGGTTTTCAGTATAATGTTACATG |
| | CGTACACGCGTCTGTACAGAAAAAA |
| | AAGAAAAATTTGAAATATAAATAAC |
| | GTTCTTAATACTAACATAACTATAA |
| | AAAAATAAATAGGGACCTAGACTTC |
| | AGGTTGTCTAACTCCTTCCTTTTCG |
| | GTTAGAGCGGATGTGGGGGGAGGGC |
| | GTGAATGTAAGCGTGACATAACTAA |
| | TTACATGACTCGAGttatttatata |
| | attcatccataccaccagttgaatg |
| | tctaccttcagctctttcatattgt |
| | tcaacaatagtataatcttcattat |
| | gtgaagtaatatccaatttaatatt |
| | aacattataagcacctggtaattga |
| | actggttttttagctttataagtag |
| | ttttaacttcagcatcataatgacc |
| | accatcttttaatttcaatctttgt |
| | ttaatttcaccttttaaagcaccat |
| | cttctggatacattctttctgatga |
| | agcttcccaacccatagtttttttt |
| | tgcataactggaccatctgatggaa |
| | aattagtacctctcaatttaacttt |
| | ataaataaattcaccatcttgtaat |
| | gatgaatcttgagtaacagtaacaa |
| | caccaccatcttcaaaattcataac |
| | tctttcccatttaaaaccttctgga |
| | aatgacaattttaaataatctggaa |
| | tatcagctggatgtttaacataagc |
| | ttttgaaccatacataaattgtggt |
| | gacaaaatatcccaagcaaatggta |
| | atggaccacctttagtaactttcaa |
| | tttagcagtttgagtaccttcatat |
| | ggtctaccttcaccttcaccttcaa |
| | tttcaaattcatgaccattaactga |
| | accttccatatgaactttaaatctc |
| | ataaattctttaataatagccatat |
| | tatcttcttcaccttttgaaaccat |
| | tatagttttttctccttgacgttaa |
| | agtatagaggtatattaacaatttt |
| | ttgttgatacttttattacatttga |
| | ataagaagtaatacaaaccgaaaat |
| | gttgaaagtattagttaaagtggtt |
| | atgcagtttttgcatttatatatct |
| | gttaatagatcaaaaatcatcgctt |
| | cgctgattaattaccccagaaataa |
| | ggctaaaaaactaatcgcattatca |
| | tcccctcgacgtactgtacatataa |
| | ccactggttttatatacagcagtac |
| | tgtacatataaccactggttttata |
| | tacagcagtcgacgtactgtacata |
| | taaccactggttttatatacagcag |
| | tactggacatataaccactggtttt |
| | atatacagcagtcgaggtaagatta |
| | gatatgAGCTCCAGCTTTTGTTCCC |
| | TTTAGTGAGGGTTAATTGCGCGCTT |
| | GGCGTAATCATGGTCATAGCTGTTT |
| | CCTGTGTGAAATTGTTATCCGCTCA |
| | CAATTCCACACAACATA |
| | (SEQ ID NO: 15). |
| | The decapitalized sequence |
| | is SEQ ID NO: 16. |

Fluorescent Protein Assay Strain Construction. V506 was grown overnight in T-media. The culture was then diluted 104-105× with T- and plated on T-plates. Single colonies were streaked on TU-, HT-, and T-plates and a streak growing on T-but not on UT- or on HT-, indicating the loss of the pMW106 and pMW3 (GSG)2rGR2 plasmids, was glycerol stocked as PBA3. This strain was transformed with plasmid HL-174-2 to generate yeast strain HL-294-7. The latter was transformed with PBA-Gib5 to generate the Y3H fluorescent reporter assay strain HL-2-4-1.

TAN-1612 Producer/Nonproducer Strain Construction. TAN-1612 producer and nonproducer strains EH-3-54-4 and EH-3-54-2 were obtained by transforming plasmids pYR342 and pYR291 or pYR342 and pRS426, respectively, into yeast strain BJ5464-NpgA (Tables 1 and 2).³⁰

Protocol for Fluorescent Protein Y3H Assay. Fresh patches of Y3H strain HL-2-4-1 were inoculated into HTU-media (1 mL) in 24-well plates (flat, clear bottom, Corning no. 3526) and placed in the shaker. After 24 h, the cells were pelleted by centrifugation at 3250 rpm and resuspended in H₂O (1 mL). Pelleting and resuspension were repeated once more. OD600 was measured by diluting 10× into H₂O, and the cells were diluted accordingly into H₂O for OD600=1 to be used as a 10× solution. A total of 20 μL of cells of OD600=1 were added to the assay plate as the last component in the assay plate (flat, clear bottom, black 96-well plate, Corning no. 3603). Each well of the assay plate contained 200 μL total liquid volume, 20 μL cells, 100 μL of 2×HTU-media of 4% raffinose and no glucose, 20 μL of 10×20% galactose aqueous solution, 20 μL of 10×CID solution of 100 μM in 10% DMSO aqueous solution, 20 μL supernatant and 20 μL purified TAN-1612 in a 10% DMSO aqueous solution,³⁰ or a 10% DMSO aqueous solution. Starting conditions in the assay plate were OD600=0.1, 2% galactose, 2% raffinose, 0% glucose, 1×HTU-, 10 μM CID, 2% DMSO. The assay plate was placed in the 30° C. shaker overnight before measuring OD600 and 620 nm fluorescence (λex=588 nm).³¹

TAN-1612 supernatants from flask cultures were obtained by inoculating TAN-1612 producing and nonproducing strains, EH-3-54-4 and EH-3-54-2, respectively, in UT-media (5 mL) in 15 mL culture tubes (Corning 352059) and placing in the shaker overnight. Overnight cultures were used to inoculate 100 mL of YPD in 500 mL conical flasks with a starting OD600 of 0.08. After 68 h, 1 mL of culture was pelleted in 1.5 mL Eppendorf tubes at 14 000 rpm, sterile filtered, and diluted 20× into H₂O to be used as a 10× stock solution.

TAN-1612 supernatants from 96-well plate cultures were obtained by inoculating 12 and 84 colonies of TAN-1612 producing and nonproducing strains, EH-3-54-4 and EH-3-54-2, respectively, in UT-media (0.5 mL) in a 96-well plate (Corning P-2 ML-SQ-C-S). The plate was covered with two layers of SealMate film (Excel Scientific, SM-KIT-BS) and placed in a shaker overnight. Overnight cultures were used to inoculate 300 μL YPD in an identical setup with a starting OD600 of 0.1 and placed in an 800 rpm shaker overnight. After 54 h, 150 μL of H₂O was added to the cultures to prevent drying of cultures due to evaporation and two new layers of Sealmate film were replaced. After 78 h, the cultures were pelleted at 3250 rpm, and the supernatant was diluted 20× into H₂O to be used as a 10× stock solution.

Protocol for Y3H Growth Assay. Fresh batches of Y3H strain PBA14 were inoculated in HT-media (1 mL) and placed in the shaker overnight. Cultures were then centrifuged at 3000 rpm for 3 min, and the pellets were resuspended with H₂O (1 mL). Pelleting and resuspension was repeated twice more. OD600 was measured, and the cells were diluted accordingly into H₂O to OD600=1 to be used as a 10× stock solution. A total of 20 μL of cells of OD600=1 were added as the last component to the assay plate (flat, clear bottom, black 96-well plate, Corning no. 3603). Each well of the assay plate contained 200 μL total liquid volume, 20 μL cells, 100 μL of 2×HT-media with 4% raffinose, 4% galactose, 0% glucose, 0.4% 5-FOA, 20 μL of 0/250 μM 10×CID in 10% DMSO aqueous solution, 20 μL of doxycycline (0/50 μM in 10% EtOH aqueous solution), and 40 μL of H2O. Starting conditions in the assay plate were OD600=0.1, 2% galactose, 2% raffinose, 0% glucose, 0.2% 5-FOA, 1×HT-, 25 μM CID, 1% DMSO, 1% EtOH. The assay plate was placed in the 30° C. shaker for 5 days with daily measurements of OD600.

Protocol for Y3H LacZ Assay. Fresh patches of Y3H strains PBA-8 and HL-260-1 through HL-260-7 were inoculated into HTU-media (1 mL) in 24-well plates (flat, clear bottom, Corning no. 3526) and place at 30° C. shaker. After 24 h, the cells were pelleted by centrifugation at 3000 rpm for 5 min and resuspended in H₂O (1 mL). Pelleting and resuspension were repeated once more. OD600 was measured, and the cells were diluted accordingly into H₂O to OD600=1 to be used as a 10× solution. A total of 20 μL of cells of OD600=1 were added to the assay plate as the last component in the assay plate (clear bottom, clear 96-well plate, Corning no. 3894). Each well of the assay plate contained 200 μL total liquid volume, 20 μL of cells, 100 μL of 2×HTU-media of 4% raffinose and no glucose, 20 μL of 10×20% galactose aqueous solution, 20 μL of 10×CID solution in 10% DMSO aqueous solution or a 10% DMSO aqueous solution, 20 μL of the tetracycline ligand in 10% EtOH aqueous solution, and 20 μL of water. Starting conditions in the assay plate are OD600=0.1, 2% galactose, 2% raffinose, 0% glucose, 1×HTU-, varying concentration CID, varying concentration of tetracycline ligand, 1% DMSO, and 1% EtOH. The assay plate was placed in the shaker overnight. OD measurements were then taken after 24 h, and the cells ere pelleted and resuspended in 100 μL of Z-buffer (60 mM Na₂HPO4, 40 mM NaH2PO4, 10 mM KCl, 2 mM MgSO₄, pH adjusted to 7, 2.7 mL/L of β-mercaptoethanol added fresh the day of the assay). The cells were pelleted again, resuspended in 100 μL of Y-PER buffer (Fisher Scientific, no. 78990), and lysed for 30 min at rt before the addition of o-nitrophenyl β-Dgalactopyranoside (ONPG) in Z-buffer (8.5 μL, 10 mg/mL). After 90 min, a Na₂CO₃ solution (1 M, 110 μL) was added to quench the reaction. After pelleting at 3000 rpm, 175 μL of the supernatant was transferred into flat clear-bottom 96-well plates (Corning no. 353072), and OD420 was measured. The limit of detection was calculated as the concentration of molecule at which the signal-to-noise ratio of the assay was >3:1.^{32,33}

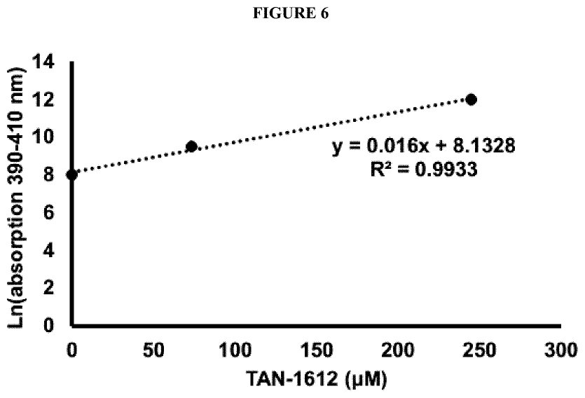

Quantification of TAN-1612 concentration in cultures. TAN-1612 cultures for quantification were obtained by inoculating two biological replicates of each of TAN-1612 producing and non-producing strains, EH-3-54-4 and EH-3-54-2 respectively, in UT-media (5 mL) in 15 mL culture tubes (Corning 352059) and placing in shaker overnight. Overnight cultures were used to inoculate 100 mL YPD in 500 mL conical flasks with a starting OD600 of 0.08. After 68 h, the 100 mL from the two biological replicates were combined and extracted with 200 mL of EtOAc in 8×50 mL falcon tubes (Corning 352098) by mixing at maximal speed in Orbital Shaker (Bellco Glass) at r.t. for 1.5 h and centrifuging at 3,300 rpm at r.t. for 15 min. The solvent of the combined organic phase was removed under reduced pressure and the sample was redissolved in MeOH (1.5 mL) and filtered (Acrodisc® Syringe Filters, 13 mm, 0.2 μm, Pall Laboratory, PTFE Membrane, 4422). The filtered sample was diluted 50× in MeOH and analyzed by Supercritical Fluid Chromatography/Mass spectrometry (SFC/MS) equipped with a photodiode array (PDA) UV-vis detector. Absorption between 390 nm to 410 nm was integrated at retention times of 0.87-1.50 min, which corresponds to TAN-1612 (MS (AI−): m/z calc. for C₂₁H₁₇O₉—: 413.09; found: 413.4 [M−H]−; MS (EI−): m/z calc. for C₂₁H₁₇O₉—: 413.09; found: 413.4 [M−H]−). The TAN-1612 concentration in the extract was calculated to be 161 μM according to the following linear function: concentration=[ln(absorption 390-410 nm)-8.1328]/0.016 (FIG. 6). The concentration of TAN-1612 in the culture is therefore estimated to be ˜60 μM, assuming a quantitative yield for the extraction process.

TABLE 5
Sequence of DHFR-B42 fusion protein

| | |
|---|---|
| DHFR-B42 | ATGGGTGCTCCTCCAAAAAAGAAGAG |
| | AAAGGTAGCTGGTATCAATAAAGAT |
| | ATCGAGGAGTGCAATGCCATCATTG |
| | AGCAGTTTATCGACTACCTGCGCAC |
| | CGGACAGGAGATGCCGATGGAAATG |
| | GCGGATCAGGCGATTAACGTGGTGC |
| | CGGGCATGACGCCGAAAACCATTCT |
| | TCACGCCGGGCCGCCGATCCAGCCT |
| | GACTGGCTGAAATCGAATGGTTTTC |
| | ATGAAATTGAAGCGGATGTTAACGA |
| | TACCAGCCTCTTGCTGAGTGGAGAT |
| | GCCTCCTACCCTTATGATGTGCCAG |
| | ATTATGCCTCTCCCGAATTGATCAG |
| | TCTGATTGCGGCGTTAGCGGTAGAT |
| | CGCGTTATCGGCATGGAAAACGCCA |
| | TGCCGTGGAACCTGCCTGCCGATCT |
| | CGCCTGGTTTAAACGCAACACCTTA |
| | AATAAACCCGTGATTATGGGCCGCC |
| | ATACCTGGGAATCAATCGGTCGTCC |
| | GTTGCCAGGACGCAAAAATATTATC |
| | CTCAGCAGTCAACCGGGTACGGACG |
| | ATCGCGTAACGTGGGTGAAGTCGGT |
| | GGATGAAGCCATCGCGGCGTGTGGT |
| | GACGTACCAGAAATCATGGTGATTG |
| | GCGGCGGTCGCGTTTATGAACAGTT |
| | CTTGCCAAAAGCGCAAAAACTGTAT |
| | CTGACGCATATCGACGCAGAAGTGG |
| | AAGGCGACACCCATTTCCCGGATTA |
| | CGAGCCGGATGACTGGGAATCGGTA |
| | TTCAGCGAATTCCACGATGCTGATG |
| | CGCAGAACTCTCACAGCTATTGCTT |
| | TGAGATTCTGGAGCGGCGGTAA |
| | (SEQ ID NO: 17) |

### Example 2. Yeast Three-Hybrid Assay

Metabolic engineering is a potentially greener and more economical approach than traditional organic synthesis for the production of a wide range of organic chemicals. As opposed to organic synthesis, metabolic engineering is capable of producing valuable chemicals from readily available and renewable carbon sources and significantly reducing the use of organic solvents and reagents.^{1,2} Traditional methods in DNA mutagenesis, advances in DNA synthesis, DNA assembly, and genome engineering are enabling high-throughput strain construction in metabolic engineering.³⁻⁸ High-throughput strain construction is necessary because not only the multigene biosynthetic pathway for the natural product but also the underlying metabolism of the host must be optimized.⁹ Assuming a biosynthetic pathway of 5 genes and 10 interacting host genes to test just 5 variants for each of these genes results already in a library size exceeding 10¹⁰. However, the throughput of the most highly employed assaying methods such as LC-MS is only ˜10² to 10³ samples per day and is thus heavily limiting in assaying strains for successful metabolic engineering.^{5,9}

The currently employed methods for assaying metabolite production in metabolic engineering are either low-throughput and general or high-throughput but not general. LC-MS methods are general but low-throughput, enabling the screening of only about 10² to 10³ samples per day.⁹ Colorimetric, fluorometric, and growth assays for molecules that are colored, fluorescent, essential for producer strain growth or could be easily converted to one of these, are high throughput but not general.¹⁰ Attempts have been made to develop assays that are both high-throughput and general, but it is yet to be determined whether or not these assays could be widely implemented.^{10,11} Versatile, general, readily implemented high-throughput assays are required to transform metabolic engineering from a field with a high potential to a field with an extensive real-world impact on the way chemicals are made.

In this Example, the Y3H assay, previously employed for various basic science applications,¹²⁻¹⁸ has been adapted to be a general, readily implemented, high-throughput versatile assay for metabolic engineering. In particular, this assay can be readily adapted to new target molecules, used as either a screen or a selection, and minimizes handling, to match varying needs and instrumentation availabilities of end users.

In this Example, the application of the Y3H assay is used to detect the biosynthesis of tetracyclines, one of the major classes of antibiotics. The engineering of the Y3H system to detect tetracyclines by synthesizing a minocycline-methotrexate chemical inducer of dimerization (CID) and cloning a LexA-TetR (tetracycline repressor) fusion protein is described.¹⁴ Reporter gene expression responds to tetracycline in this system by competitive binding of tetracycline and the CID to the LexA-TetR protein fusion (FIG. 1). This assay can report the presence of tetracyclines using various reporter gene outputs, enabling the use of the assay both as a screen and a selection to match the needs and instrumentation of a variety of end users. The applicability of this assay to metabolic engineering is shown through the differentiation between producer and nonproducer strains of the natural product tetracycline, TAN-1612.

For a Y3H system detecting tetracyclines, a Y3H strain encoding LexA-TetR and DHFR-B42 fusion proteins is cloned and synthesized a minocycline-methotrexate (Min-Mtx) CID (FIG. 1). This Y3H system is based on well-studied components, namely, the LexA DNA-binding domain (DBD) and B42 activation domain (AD),³⁴ respectively, introduced by Brent and co-workers,^{14,27,35,36} as well as the DHFR-methotrexate and TetR-tetracycline interactions. The latter two are highly characterized receptor-ligand interactions of picomolar affinity,³⁷ and the methotrexate-DFHR interaction has been used numerous times.^{27,35} LexA-TetR fusion proteins of TetR variants that were highly studied were used, such as the variants from classes B and D,³⁷⁻³⁹ as well TetR variants from the much less studied TetR classes A,^{40,41} C,^{42,43} E,^{44,45} G,⁴⁶ and H.⁴⁷⁻⁵⁰ TetR and LexA form stable homodimers, with each of the TetR monomers binding tetracyclines and LexA binding DNA as a dimer.^{38,51} Thus, it is possible that the monomer ratio of TetR/LexA in the LexA-TetR fusion protein is unequal to 1, affecting accordingly the local concentrations of the Min-Mtx CID and DHFR-B42 as well. In any case, increased levels of tetracyclines are expected to outcompete the CID from the LexA-TetR protein fusion and lower reporter protein expression (FIG. 1).

The design and synthesis of the Min-Mtx CID is based on the wealth of the structure-activity relationship (SAR) data for the interaction of tetracyclines with TetR, as well as previously reported CIDs.^{27,35} It is known from inspection of the high-resolution structure of TetR bound to tetracycline derivatives as well as biochemical characterization that tetracyclines can be derivatized at the D ring without disrupting binding to TetR (SCHEME 1).^{37,38,52} Moreover, the FDA-approved analogue tigecycline and the clinical candidates eravacycline and TP-271 are all 9-amidotetracyclines;^{53,54} thus, synthesis of a 9-amidotetracycline CID was logical. Specifically, 9-aminominocycline was chosen as the starting material because of its high acid stability relative to tetracycline as well as its commercial availability.⁵⁵ The design and synthesis of the Mtx component of the Min-Mtx CID was based on previously reported methotrexate CIDs (Scheme 1).^{27,35}

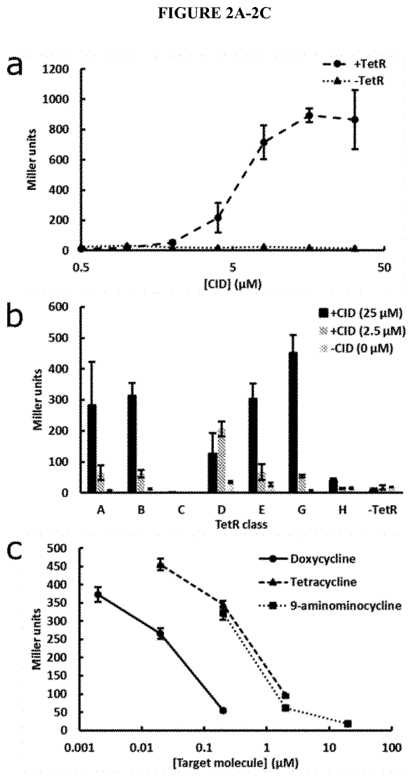

With the Y3H strains and Min-Mtx CID in hand, the functionality of the Y3H system was verified and the optimal CID concentration and TetR variant were determined to further use in the assay. First, it was confirmed that the reporter gene output in this system is dependent upon the presence of the CID and determined the desired range of CID concentration to use in the assay (FIG. 2A). Then the candidates from each of the TetR classes were tested as protein receptors and it was observed that the Y3H system is responsive to the CID with all known TetR classes except for the TetR(C) variant used (FIG. 2B). Given that TetR(B) and TetR(G) showed the best response to the CID and to maintain consistency with the highly studied TetR(B),³⁷⁻³⁹ further experiments focused on TetR(B).

This assay can differentiate sub-μM concentrations of various tetracyclines (FIG. 2C), key for its applicability to differentiate between high and low producer strains. This Y3H system differentiates doxycycline, tetracycline, and 9-NH2-minocycline concentrations in the range 2-200 nM, 0.02-2 μM, and 0.2-20 μM, respectively. The limit of detection in this Y3H assay is ≤0.2 μM for doxycycline and ≤2 μM for tetracycline and 9-NH2-minocycline.³²

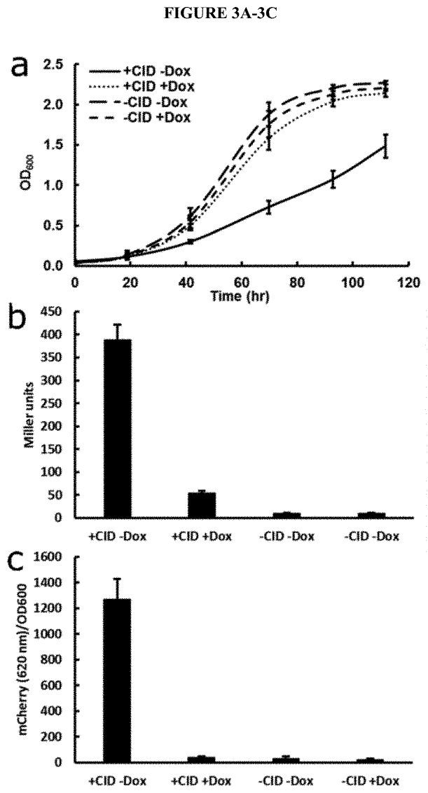

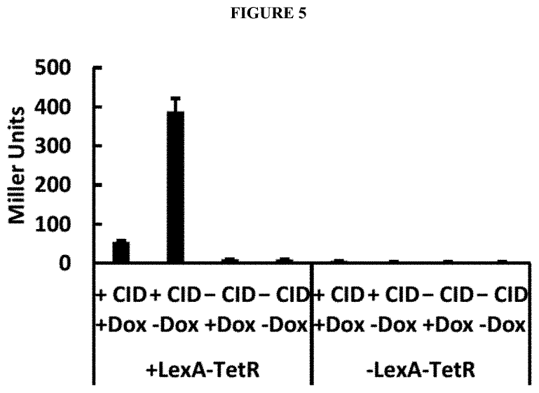

After confirming the functionality of the Y3H system for tetracyclines, it is shown to report the presence of tetracyclines with a modular output, key for enabling versatile use by various end users. Using a URA3 reporter gene and growing the yeast in the presence of 5-fluorouracil (5-FOA), yeast growth is shown to be doxycycline-dependent in the presence, but not in the absence of the Min-Mtx CID (FIG. 3A). Additionally, the Y3H assay for tetracyclines can be used as a colorimetric (FIG. 3B) as well as a fluorometric assay, by employing a LacZ or and mCherry reporter gene, respectively (FIG. 3B, C). As expected, while reporter gene activity is dependent on CID and doxycycline in the Y3H strain, such dependence is not detected in a control strain excluding the eDFHR-AD fusion protein (FIG. 5).

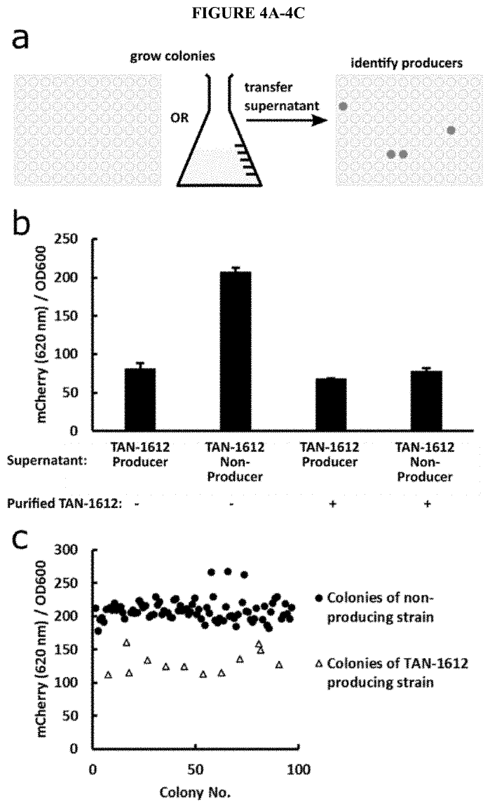

The applicability of the Y3H assay to metabolic engineering is demonstrated by differentiating between producer and nonproducer strains of TAN-1612 (FIG. 4A).³° For the TAN-1612 producer strain, the S. cerevisiae strain EH-3-54-4 was used, encoding all four genes of the TAN-1612 biosynthetic pathway as previously reported by Tang and co-workers and producing ˜60 μM TAN-1612.³⁰ The nonproducer control used, S. cerevisiae strain EH-3-54-2, does not encode the polyketide synthase AdaA and has no measurable production of TAN-1612. The Y3H assay for tetracyclines can differentiate between supernatants of producer and nonproducer strains of TAN-1612 (FIG. 4B). When purified TAN-1612 is spiked into nonproducer supernatants, the resulting Y3H signal resembles the signal obtained with producer supernatant (FIG. 4B). These results agree with the hypothesis that TAN-1612, present in the producer strain supernatant but not in the nonproducer strain supernatant, outcompetes the CID from the LexA-TetR fusion protein.

The Y3H assay for tetracyclines also differentiates TAN-1612 producer colonies from a nonproducing population cultured in a 96-well plate (FIG. 4C). The 12 producer colonies gave an average mCherry fluorescence/OD600 signal of 132 with a standard deviation of 17, and the 84 nonproducer colonies gave an average mCherry fluorescence/OD600 signal of 210 with a standard deviation of 16. Thus, assuming normal distribution there is less than a 2.5% overlap in the Y3H signal from producer and nonproducer colonies.

The Y3H assay for tetracyclines is advantageous compared to the state-of-the-art methods for tetracycline detection in terms of throughput, cost, instrumentation requirements, and limit of detection. Being readily performed in 96-well microtiter plates, the Y3H assay throughput is >1000 samples/hour when measured by a standard laboratory plate reader (Infinite-M200). By comparison, the throughput of the previous state of-the-art HPLC-based methods for tetracycline detection is <10 samples/hour.^{33,57} The use of a plate reader to measure the Y3H assay output is also advantageous in terms of cost to state-of-the-art chromatography-based methods such as HPLC and even more so, LC-MS. The price difference is both in the initial investment in instrumentation, as spectrophotometers are cheaper than LC instruments, and in the reagent costs of 96-well plates vs LC-grade solvents. In terms of limit of detection, the Y3H assay has a limit of detection of ≤200 nM for doxycycline (FIG. 2C), which is better than the state-of the-art HPLC limit of detection of ≥4.5 μM for doxycycline.^{33,57} The low limit of detection of this assay exhibits enables it to differentiate producer from nonproducer strains even in low titers of initial strain development (FIG. 4, ˜60 μM). Differentiating between production levels in higher titer ballparks in later stages of producer strain optimization would simply require a higher dilution factor of supernatants into the Y3H strain media. Future generations of this Y3H assay might include TetR mutants that are more specific and have a lower detection limit to tetracycline derivatives of interest.

The Y3H assay for metabolic engineering also compares well with the recently published FP assay⁵⁸ because of two key differences in setup requirements and output. First, since the Y3H assay does not require the purification of the protein receptor, it is used much more readily to screen for the ultimate protein receptor for the assay. Thus, it is shown here the first comparison of all TetR classes for binding of a small molecule in an attempt to identify the best TetR variant for differentiating between + and −CID (FIG. 2B). Further optimization of a protein receptor for the assay of a specific target molecule is readily enabled in the Y3H assay simply by cloning the protein variants into the same plasmid backbone and transforming into yeast. The versatility in choice of output in the Y3H assay is a key enabling factor for the advantages the Y3H assay has over the FP assay since it makes the assay accessible to a wider range of users. The fluorescent output in the Y3H assay enables measurements with a standard laboratory plate reader, eliminating the requirement for anisotropy capacities (FIG. 3C). The LacZ output could also be measured by a standard plate reader as well as by the naked eye (FIG. 3B).

If the Y3H system and the metabolic pathway for the small molecule of interest are cloned into the same strain, colonies can be assayed for production even more directly, increasing throughput and minimizing handling. The Y3H assay can then enable screening colonies for production of the target molecule directly on an agar plate without the need for further handling of the colonies in microtiter plates. The agar plate screen could be performed with the naked eye or a simple camera by using X-Gal plates and the LacZ reporter or fluorescent camera/microscope by using the mCherry reporter. Finally, the Y3H assay throughput could be vastly increased by selecting for high-producing strains en masse in flask or fermenter culturing using the growth assay or fluorescence assisted cell sorting (FACS). Such en masse screening methods not only greatly enhance the throughput but also minimize the labor, instrumentation, cost, and time required to obtain results. The Y3H assay for tetracyclines can report the presence of a target molecule with any of the above-mentioned outputs (FIG. 3), enabling metabolic engineering applications for a range of end users with varying needs and instrumentation availabilities.

While a TetR-tetO-based assay could conceivably be developed for the metabolic engineering of tetracycline derivatives,⁵⁹ this TetR-based Y3H assay is advantageous for two main reasons. For one, the Y3H assay can potentially be applied to a greater variety of tetracycline derivatives as TetR binding to the tetracycline suffices and there is no demand for a TetR conformational change. By comparison, signal transduction in the TetR-tetO system is dependent on coupling between tetracycline binding and a conformational change in TetR.^{26,38} It has been hypothesized that high-affinity binding can be a necessary but not sufficient property for TetR induction in the TetR-TetO system.⁶⁰ In practice, while it was possible to detect binding to a tetracycline analogue, 4-de(dimethylamino)-anhydrotetracycline, no induction was detected using a TetR-TetO-based assay.³⁷ Thus, the use of TetR in a Y3H system can facilitate the evolvement of TetR mutants with specific binding to tetracycline derivatives of the interest in future generations of the assay. Furthermore, the Y3H system for metabolic engineering can be readily modified to apply for many metabolites of interest beyond tetracyclines, as detailed below. In contrast, the TetR-TetO system is much more limited in the scope of molecules it could be applied to, namely, transcriptional regulators.⁶¹

A Y3H system as described here for the metabolic engineering of tetracycline derivatives can be designed for any target molecule with a receptor. Given T, a desirable target, PR, a protein receptor for T, and T′, a readily derivatizable analogue of T, two new components need to be generated for a Y3H assay to detect biosynthesis of T. The first is a Mtx-T′ CID, and the second is a LexA-PR fusion protein (FIG. 1). This approach is especially amenable for the development of assays for molecules of pharmacological (i.e., therapeutic molecule) of interest. The reason is that such molecules often have a known protein target as well as an established derivatization chemistry and SAR that greatly support the design and synthesis of a CID. More broadly, building from early reports of chemical complementation,^{17,62,63} this work advances the enabling technology of the Y3H system for the emerging field of synthetic biology for handling the diversity of engineering multigene pathways and assaying for chemistry not natural to the cell.

### REFERENCES

(1) Fasciotti, M. (2017) Perspectives for the use of biotechnology in green chemistry applied to biopolymers, fuels and organic synthesis: from concepts to a critical point of view. Sustainable Chem. Pharm. 6, 82-89.
(2) Keasling, J. D. (2010) Manufacturing Molecules Through Metabolic Engineering. Science 330, 1355-1358.
(3) Chao, R., Yuan, Y., and Zhao, H. (2014) Recent advances in DNA assembly technologies. FEMS Yeast Res. 15, 1-9.
(4) Kosuri, S., and Church, G. M. (2014) Large-scale de novo DNA synthesis: technologies and applications. Nat. Methods 11, 499-507.
(5) Jullesson, D., David, F., Pfleger, B., and Nielsen, J. (2015) Impact of synthetic biology and metabolic engineering on industrial production of fine chemicals. Biotechnol. Adv. 33, 1395-1402.
(6) Richardson, S. M., Mitchell, L. A., Stracquadanio, G., Yang, K., Dymond, J. S., DiCarlo, J. E., Lee, D., Huang, C. L. V., Chandrasegaran, S., Cai, Y., Boeke, J. D., and Bader, J. S. (2017) Design of a synthetic yeast genome. Science 355, 1040-1044.
(7) Wang, H. H., Isaacs, F. J., Carr, P. A., Sun, Z. Z., Xu, G., Forest, C. R., and Church, G. M. (2009) Programming cells by multiplex genome engineering and accelerated evolution. Nature 460, 894-898.
(8) Doudna, J. A., and Charpentier, E. (2014) The new frontier of genome engineering with CRISPR-Cas9. Science 346, 1258096-1258096.
(9) Rogers, J. K., Taylor, N. D., and Church, G. M. (2016) Biosensor-based engineering of biosynthetic pathways. Curr. Opin. Biotechnol. 42, 84-91.
(10) Schallmey, M., Frunzke, J., Eggeling, L., and Marienhagen, J. (2014) Looking for the pick of the bunch: high-throughput screening of producing microorganisms with biosensors. Curr. Opin. Biotechnol. 26, 148-154.
(11) Feng, J., Jester, B. W., Tinberg, C. E., Mandell, D. J., Antunes, M. S., Chari, R., Morey, K. J., Rios, X., Medford, J. I., Church, G. M., Fields, S., and Baker, D. (2015) A general strategy to construct small molecule biosensors in eukaryotes. eLife 4, 1.
(12) Licitra, E. J., and Liu, J. O. (1996) A three-hybrid system for detecting small ligand-protein receptor interactions. Proc. Natl. Acad. Sci. U.S.A 93, 12817-12821.
(13) Belshaw, P. J., Ho, S. N., Crabtree, G. R., and Schreiber, S. L. (1996) Controlling protein association and subcellular localization with a synthetic ligand that induces heterodimerization of proteins. Proc. Natl. Acad. Sci. U.S.A 93, 4604-4607.
(14) Golemis, E. A., Serebriiskii, I., Finley, R. L., Kolonin, M. G., Gyuris, J., and Brent, R. (2008) Interaction Trap/Two-Hybrid System to Identify Interacting Proteins. Curr. Protoc. Cell Biol. 53, 17.13.11-17.13.35.
(15) Bernstein, D. S., Buter, N., Stumpf, C., and Wickens, M. (2002) Analyzing mRNA-protein complexes using a yeast three-hybrid system. Methods 26, 123-141.
(16) Henthorn, D. C., Jaxa-Chamiec, A. A., and Meldrum, E. (2002) A GAL4-based yeast three-hybrid system for the identification of small molecule-target protein interactions. Biochem. Pharmacol. 63, 1619-1628.
(17) Baker, K., Bleczinski, C., Lin, H., Salazar-Jimenez, G., Sengupta, D., Krane, S., and Cornish, V. W. (2002) Chemical complementation: a reaction-independent genetic assay for enzyme catalysis. Proc. Natl. Acad. Sci. U.S.A 99, 16537-16542.
(18) Fields, S., and Song, O. K. (1989) A Novel Genetic System to Detect Protein Protein Interactions. Nature 340, 245-246.
(19) Morita, T., and Takegawa, K. (2004) A simple and efficient procedure for transformation of Schizosaccharomyces pombe. Yeast 21, 613-617.
(20) Gibson, D. G., Young, L., Chuang, R. Y., Venter, J. C., Hutchison, C. A., 3rd, and Smith, H. O. (2009) Enzymatic assembly of DNA molecules up to several hundred kilobases. Nat. Methods 6, 343-345.
(21) Green, S. R., and Moehle, C. M. (2001) Media and Culture of Yeast. Curr. Protoc. Cell Biol. 4, 1.6.1-1.6.12.
(22) Krishnan, L., Sum, P.-E., Daigneault, S., Bernatchez, M., Pilcher, A. S., Horne, J. M., Tuper, A. J., Mccauley, III, J. J., and Michaud, A. P. (2006) Methods of purifying tigecycline, U.S. Patent 2007/0049561 A1, Mar. 1, 2017.
(23) Nagy, A., Szoke, B., and Schally, A. V. (1993) Selective Coupling of Methotrexate to Peptide-Hormone Carriers through a Gamma-Carboxamide Linkage of Its Glutamic-Acid Moiety-Benzotriazol-1-Yloxytris(Dimethylamino) Phosphonium Hexafluorophosphate Activation in Salt Coupling. Proc. Natl. Acad. Sci. U.S.A 90, 6373-6376.
(24) Castex, U., Lalanne, C., Mouchet, P., Lemaire, M., and Lahana, R. (2005) Regioselective synthesis of peptidic derivatives and glycolamidic esters of methotrexate. Tetrahedron 61, 803-812.
(25) Reichheld, S. E., and Davidson, A. R. (2006) Two-way interdomain signal transduction in tetracycline repressor. J. Mol. Biol. 361, 382-389.
(26) Reichheld, S. E., Yu, Z., and Davidson, A. R. (2009) The induction of folding cooperativity by ligand binding drives the allosteric response of tetracycline repressor. Proc. Natl. Acad. Sci. U.S.A 106, 22263-22268.
(27) Abida, W. M., Carter, B. T., Althoff, E. A., Lin, H., and Cornish, V. W. (2002) Receptor-dependence of the transcription read-out in a small-molecule three-hybrid system. ChemBioChem 3, 887-895.
(28) Harton, M. D., Wingler, L. M., and Cornish, V. W. (2013) Transcriptional regulation improves the throughput of three-hybrid counter selections in Saccharomyces cerevisiae. Biotechnol. J. 8, 1485-1491.
(29) Keppler-Ross, S., Noffz, C., and Dean, N. (2008) A New Purple Fluorescent Color Marker for Genetic Studies in Saccharomyces cerevisiae and Candida albicans. Genetics 179, 705-710.
(30) Li, Y. R., Chooi, Y. H., Sheng, Y. W., Valentine, J. S., and Tang, Y. (2011) Comparative Characterization of Fungal Anthracenone and Naphthacenedione Biosynthetic Pathways Reveals an alpha-Hydroxylation-Dependent Claisen-like Cyclization Catalyzed by a Dimanganese Thioesterase. J. Am. Chem. Soc. 133, 15773-15785.
(31) Ostrov, N., Jimenez, M., Billerbeck, S., Brisbois, J., Matragrano, J., Ager, A., and Cornish, V. W. (2017) A modular yeast biosensor for low-cost point-of-care pathogen detection. Science Advances 3, e1603221.
(32) ICH Expert Working Group. (2005) Validation of analytical procedures: text and methodology Q2 (R1), in International Conference on Harmonization, Geneva, Switzerland.
(33) Monser, L., and Darghouth, F. (2000) Rapid liquid chromatographic method for simultaneous determination of tetracyclines antibiotics and 6-Epi-doxycycline in pharmaceutical products using porous graphitic carbon column. J. Pharm. Biomed. Anal. 23, 353-362.
(34) Ruden, D. M., Ma, J., Li, Y., Wood, K., and Ptashne, M. (1991) Generating Yeast Transcriptional Activators Containing No Yeast Protein Sequences. Nature 350, 250-252.
(35) Lin, H., Abida, W. M., Sauer, R. T., and Cornish, V. W. (2000) Dexamethasone-Methotrexate: An Efficient Chemical Inducer of Protein Dimerization In Vivo. J. Am. Chem. Soc. 122, 4247-4248.
(36) Gyuris, J., Golemis, E., Chertkov, H., and Brent, R. (1993) Cdi1, a human G1 and S phase protein phosphatase that associates with Cdk2. Cell 75, 791-803.
(37) Lederer, T., Kintrup, M., Takahashi, M., Sum, P. E., Ellestad, G. A., and Hillen, W. (1996) Tetracycline analogs affecting binding to Tn10-Encoded Tet repressor trigger the same mechanism of induction. Biochemistry 35, 7439-7446.
(38) Hinrichs, W., Kisker, C., Duvel, M., Muller, A., Tovar, K., Hillen, W., and Saenger, W. (1994) Structure of the Tet repressor tetracycline complex and regulation of antibiotic resistance. Science 264, 418-420.
(39) Saenger, W., Hinrichs, W., Orth, P., Schnappinger, D., and Hillen, W. (2000) Structural basis of gene regulation by the tetracycline inducible Tet repressor-operator system. Nat. Struct. Biol. 7, 215-219.
(40) Waters, S. H., Rogowsky, P., Grinsted, J., Altenbuchner, J., and Schmitt, R. (1983) The tetracycline resistance determinants of RP1 and Tn1721: nucleotide sequence analysis. Nucleic Acids Res. 11, 6089-6105.
(41) Rhodes, G., Parkhill, J., Bird, C., Ambrose, K., Jones, M. C., Huys, G., Swings, J., and Pickup, R. W. (2004) Complete Nucleotide Sequence of the Conjugative Tetracycline Resistance Plasmid pFBAOT6, a Member of a Group of IncU Plasmids with Global Ubiquity. Appl. Environ. Microbiol. 70, 7497-7510.
(42) Brow, M. A. D., Pesin, R., and Sutcliffe, J. G. (1985) The Tetracycline Repressor of Psc101. Mol. Biol. Evol. 2, 1-12.
(43) Bernardi, A., and Bernardi, F. (1984) Complete sequence of pSC101. Nucleic Acids Res. 12, 9415-9426.
(44) Tovar, K., Ernst, A., and Hillen, W. (1988) Identification and Nucleotide-Sequence of the Class-E Tet Regulatory Elements and Operator and Inducer Binding of the Encoded Purified Tet Repressor. Mol. Gen. Genet. 215, 76-80.
(45) Gao, F., Webb, H. E., Bugarel, M., den Bakker, H. C., Nightingale, K. K., Granier, S. A., Scott, H. M., and Loneragan, G. H. (2016) Carbapenem-Resistant Bacteria Recovered from Faeces of Dairy Cattle in the High Plains Region of the USA. PLoS One 11, e0147363.
(46) Liu, P., Li, P., Jiang, X., Bi, D., Xie, Y., Tai, C., Deng, Z., Raj akumar, K., and Ou, H. Y. (2012) Complete Genome Sequence of Klebsiella pneumoniae subsp. pneumoniae HS11286, a Multidrug-Resistant Strain Isolated from Human Sputum. J. Bacteriol. 194, 1841-1842.
(47) Hansen, L. M., Mcmurry, L. M., Levy, S. B., and Hirsh, D. C. (1993) A New Tetracycline Resistance Determinant, Tet H, from Pasteurella-Multocida Specifying Active Efflux of Tetracycline. Antimicrob. Agents Chemother. 37, 2699-2705.
(48) Kehrenberg, C., Tham, N. T. T., and Schwarz, S. (2003) New Plasmid-Borne Antibiotic Resistance Gene Cluster in Pasteurella multocida. Antimicrob. Agents Chemother. 47, 2978-2980.
(49) Mendez, B., Tachibana, C., and Levy, S. B. (1980) Heterogeneity of tetracycline resistance determinants. Plasmid 3, 99-108.
(50) Hillen, W., and Berens, C. (1994) Mechanisms Underlying Expression of TN10 Encoded Tetracycline Resistance. Annu. Rev. Microbiol. 48, 345-369.
(51) Mohana-Borges, R., Pacheco, A. B. F., Sousa, F. J. R., Foguel, D., Almeida, D. F., and Silva, J. L. (2000) LexA Repressor Forms Stable Dimers in Solution. J. Biol. Chem. 275, 4708-4712.
(52) Kisker, C., Hinrichs, W., Tovar, K., Hillen, W., and Saenger, W. (1995) The Complex Formed between Tet Repressor and Tetracycline-Mg2+ Reveals Mechanism of Antibiotic-Resistance. J. Mol. Biol. 247, 260-280.
(53) Greer, N. D. (2006) Tigecycline (Tygacil): the first in the glycylcycline class of antibiotics. Proc. (Bayl. Univ. Med. Cent.) 19, 155-161.
(54) Liu, F., and Myers, A. G. (2016) Development of a platform for the discovery and practical synthesis of new tetracycline antibiotics. Curr. Opin. Chem. Biol. 32, 48-57.
(55) Rogalski, W. (1985) Chemical Modification of the Tetracyclines, in The Tetracyclines (Hlavka, J. J., and Boothe, J. H., Eds.), pp 179-316, Springer Berlin Heidelberg, Berlin, Heidelberg.
(56) Zhang, X., and Bremer, H. (1995) Control of the Escherichia coli rrnB P1 promoter strength by ppGpp. J. Biol. Chem. 270, 11181-11189.
(57) Kogawa, A. C., and Salgado, H. R. N. (2013) Quantification of Doxycycline Hyclate in Tablets by HPLC-UV Method. J. Chromatogr. Sci. 51, 919-925.
(58) Ng, Y. Z., Baldera-Aguayo, P. A., and Cornish, V. W. (2017) Fluorescence Polarization Assay for Small Molecule Screening of FK506 Biosynthesized in 96-Well Microtiter Plates. Biochemistry 56, 5260-5268.
(59) Gossen, M., and Bujard, H. (1992) Tight Control of Gene-Expression in Mammalian-Cells by Tetracycline-Responsive Promoters. Proc. Natl. Acad. Sci. U.S.A 89, 5547-5551.
(60) Scholz, O., Kostner, M., Reich, M., Gastiger, S., and Hillen, W. (2003) Teaching TetR to recognize a new inducer. J. Mol. Biol. 329, 217-227.
(61) Ramos, J. L., Martinez-Bueno, M., Molina-Henares, A. J., Teran, W., Watanabe, K., Zhang, X., Gallegos, M. T., Brennan, R., and Tobes, R. (2005) The TetR Family of Transcriptional Repressors. Microbiol. Mol. Biol. Rev. 69, 326-356.
(62) Lin, H., Tao, H., and Cornish, V. W. (2004) Directed evolution of a glycosynthase via chemical complementation. J. Am. Chem. Soc. 126, 15051-15059.
(63) Peralta-Yahya, P., Carter, B. T., Lin, H., Tao, H., and Cornish, V. W. (2008) High-throughput selection for cellulase catalysts using chemical complementation. J. Am. Chem. Soc. 130, 17446-17452.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

The contents of all figures and all references, patents and published patent applications and Accession numbers cited throughout this application are expressly incorporated herein by reference.

1. A genetically-engineered cell expressing:
(a) a first fusion protein comprising (i) a DNA-binding domain and (ii) a protein or fragment thereof that binds to a molecule; and
(b) a second fusion protein comprising (i) an activation domain and (ii) a protein or fragment thereof that binds to methotrexate.

2. The genetically-engineered cell of claim 1, further comprising a nucleic acid comprising (i) a DNA-binding site that binds to the DNA-binding domain of the first fusion protein and (ii) a reporter gene.

3. The genetically-engineered cell of claim 1 or 2, further comprising a chemical inducer of dimerization (CID), wherein the CID comprises (i) a ligand that binds to the protein or fragment thereof of the first protein that binds to the molecule and (ii) methotrexate;

4. The genetically-engineered cell of any one of claims 1-3, wherein the genetically-engineered cell is selected from the group consisting of a mammalian cell, a plant cell, a bacterial cell and a fungal cell.

5. The genetically-engineered cell of claim 4, wherein the fungal cell is a yeast cell.

6. The genetically-engineered cell of any one of claims 1-5, wherein the molecule is a therapeutic molecule.

7. The genetically-engineered cell of claim 6, wherein the therapeutic molecule is selected from the group consisting of a peptide, a protein or portion thereof and a small molecule.

8. The genetically-engineered cell of claim 7, wherein the therapeutic molecule is a small molecule.

9. The genetically-engineered cell of claim 8, wherein the small molecule is tetracycline or a derivative thereof.

10. The genetically-engineered cell of any one of claims 1-9, wherein:
(a) the DNA-binding domain is LexA or a DNA-binding domain thereof;
(b) the protein or fragment thereof that binds to the molecule is a tetracycline receptor (TetR) or a variant thereof;
(c) the activation domain is B42 or an activation domain thereof; and/or
(d) the protein or fragment thereof that binds to methotrexate is dihydrofolate reductase (DHFR);

11. The genetically-engineered cell of claim 10, wherein the TetR or variant thereof is selected from the group consisting of TetR(A), TetR(B), TetR(C), TetR(D), TetR(E), TetR(G), TetR(H) or a variant thereof.

12. The genetically-engineered cell of any one of claims 1-11, wherein:
(a) the first fusion protein is encoded by a nucleotide sequence that is at least about 90% homologous to a sequence of any one of SEQ ID NOs: 1-7;
(b) the protein or fragment thereof that binds to the molecule is encoded by a nucleotide sequence that is at least about 90% homologous to a sequence of any one of SEQ ID NOs: 8-14; and/or
(c) the second fusion protein is encoded by a nucleotide sequence that is at least 90% homologous to the sequence of SEQ ID NO: 17.

13. The genetically-engineered cell of claim 2, wherein the reporter gene is a conditionally-essential gene or encodes LacZ or a fluorescent protein.

14. The genetically-engineered cell of claim 13, wherein the conditionally-essential gene is URA3.

15. The genetically-engineered cell of claim 2, wherein the nucleic acid comprises a nucleotide sequence that is at least about 90% homologous to a sequence of any one of SEQ ID NOs: 15-16.

16. The genetically-engineered cell of claim 2, wherein dimerization of the first fusion protein and the second fusion protein induces expression of the reporter gene.

17. The genetically-engineered cell of claim 3, wherein the ligand of the CID that binds to the protein or fragment thereof of the first fusion protein that binds to the molecule comprises a tetracycline derivative.

18. An assay for detecting the presence of a molecule comprising:
(a) contacting the genetically-engineered cell of claim 2 with:
(1) a chemical inducer of dimerization (CID), wherein the CID comprises
(i) a ligand that binds to the protein or fragment thereof of the first fusion protein that binds to the molecule and (ii) methotrexate; and
(2) a sample; and
(b) detecting the expression of the reporter gene,
wherein the CID dimerizes the first fusion protein and the second fusion protein to induce expression of the reporter gene,
wherein reduced expression of the reporter gene as compared to a reference control indicates the presence of the therapeutic molecule in the sample.

19. The assay of claim 18, wherein the sample is supernatant from a culture of a cell that produces and secretes the molecule.

20. The assay of any one of claims 18-19, wherein the molecule is a therapeutic molecule.

21. The assay of claim 20, wherein the therapeutic molecule is selected from the group consisting of a peptide, a protein or portion thereof and a small molecule.

22. The assay of claim 21, wherein the small molecule is tetracycline or a derivative thereof.

23. The assay of claim 21, wherein the ligand of the CID that binds to the protein or fragment thereof that binds to the therapeutic molecule of the first fusion protein comprises a tetracycline derivative.

24. The assay of any one of claims 18-23, wherein:
(a) the DNA-binding domain is LexA or a DNA-binding domain thereof;
(b) the protein or fragment thereof that binds to the molecule is a tetracycline receptor (TetR) or a variant thereof;
(c) the activation domain is B42 or an activation domain thereof;
(d) the protein or fragment thereof that binds to methotrexate is dihydrofolate reductase (DHFR);
(e) the DNA-binding site is a binding site for LexA; and/or
(f) the reporter gene is a conditionally-essential gene or encodes LacZ or a fluorescent protein.

25. The assay of any one of claims 18-24, wherein the molecule competes with the CID for binding to the protein or fragment thereof of the first fusion protein that binds to the molecule.

26. A kit for detecting the presence of a molecule comprising:
(a) a genetically-engineered cell comprising a nucleic acid comprising (i) a DNA-binding site and (ii) a reporter gene;
(b) a nucleic acid encoding a first fusion protein comprising (i) a DNA-binding domain that binds to the DNA-binding site and (ii) a protein or fragment thereof that binds to a molecule; and/or
(c) a nucleic acid encoding a second fusion protein comprising (i) an activation domain and (ii) a protein or fragment thereof that binds to methotrexate.

27. A kit for detecting the presence of a molecule comprising a genetically-engineered cell of any one of claims 1-3.

28. The kit of claim 26 or 27 further comprising a chemical inducer of dimerization (CID), wherein the CID comprises (i) a ligand that binds to the protein or fragment thereof that binds to the molecule of the first fusion protein and (ii) methotrexate.

29. The kit of any one of claims 26-28, wherein the molecule is a therapeutic molecule.
